(19) United States
(12) Patent Application Publication
(10) Pub. No.: US 2005/0281815 A1
(43) Pub. Date: Dec. 22, 2005

(54) CD40 splice variants and their uses

(76) Inventors: Dani Eshel, Pardes-Hana Karkur (IL); Galit Rotman, Herzliya (IL); Kinneret Savitsky, Tel-Aviv (IL); Amir Toporik, Azur (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/069,856

(22) Filed: Feb. 28, 2005

Related U.S. Application Data
(60) Provisional application No. 60/547,422, filed on Feb. 26, 2004.
(60) Provisional application No. 60/647,822, filed on Jan. 31, 2005.

(51) Int. Cl. A61K 39/395; C07H 21/04; C12P 21/06; C12N 5/06; C07K 16/28

(52) U.S. Cl. 424/144.1; 530/388.22; 435/69.1; 435/334; 435/320.1; 536/23.53; 435/252.3

(57) ABSTRACT

Disclosed are CD40 splice variant polypeptides and methods of using the CD40 splice variant polypeptides, including in treatment for transplantation.

### RELATED APPLICATIONS

The application claims priority to U.S. Ser. No. 60/547,422, filed Feb. 26, 2004, and U.S. Ser. No. 60/647,822, filed Jan. 31, 2005. The contents of these applications are incorporated herein by reference in their entireties.

### FIELD OF THE INVENTION

The present invention relates to CD40 splice variant polypeptides and methods of using the CD40 splice variant polypeptides, including in treatment for transplantation.

### BACKGROUND OF THE INVENTION

CD40 was originally described as a receptor responsible for the activation and differentiation of B-lymphocytes. This receptor engages to its ligand (CD154, also named “CD40-L”; CD40 receptor is sometimes referred to as “CD40-R”), promoting cell survival and costimulatory protein expression necessary for interaction with T-lymphocytes. Thus, interaction of B- and T-cells via the CD40-CD154 system allows mutual activation, with B-cells secreting antibodies and T-cells becoming effector cells producing cytokines.

However, the CD40-CD154 system has wider implications than just activation of B- and T-lymphocytes. CD40 is also expressed by migratory immune cells, such as macrophages and dendritic cells, which present antigens and activate T-lymphocytes. Engagement of CD40 by T-lymphocyte CD154 activates these immune cells to express new immune modulators, such as the cytokines IL-1, IL-12 and TNF. Additionally, non-hematopoietic cells, including fibroblasts, endothelial cells, smooth muscle cells and some epithelial cells, constitutively display CD40 on their surface, and that this expression is upregulated following exposure to IFN. CD40 signaling in non-hematopoietic cells via CD154 results in initiation of cellular functions, such as synthesis of pro-inflammatory cytokines. CD40 engagement on endothelial and vascular smooth muscle cells induces synthesis of matrix matalloproteinases (MMP), which degrades collagens and other connective tissue proteins crucial for the stability of atherosclerotic plaques and their fibrous caps.

Initially, it was thought that CD154 is expressed only on the surface of T-lymphocytes after their activation. However, CD154 was also found to be expressed by eosinophils and mast. In addition, human platelets have pre-formed CD154 inside them. Once activated by thrombin or other mediators, platelet internal stores of CD154 are exported to the surface where some is secreted. Several other cell types are now known to have CD154 stored within. These include macrophages, B-lymphocytes, endothelial cells and smooth muscle cells.

A number of pathological processes of chronic inflammatory diseases in humans, and several experimental animal models of chronic inflammation, were shown to be dependent upon or involve the CD40-CD154 system (Xu Y, Song G., J Biomed Sci. 2004 July-August; 11(4):426-38; Chitnis T, Khoury S J., J Allergy Clin Immunol. 2003 November; 112 (5):837-49; Flavell R A. Curr Top Microbiol Immunol. 2002; 266:1-9.) These include graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, as well as hematological malignancies and other cancers (Tong A W, Stone M J. Cancer Gene Ther. 2003 January; 10(1):1-13; Flavell R A. Curr Top Microbiol Immunol. 2002; 266:1-9).

CD154, and specifically signaling through CD40-CD154 interactions, was found to be important in the process of organ transplant rejection. For example, treatment with anti-CD154 antibodies was found to inhibit allograft rejection in different organ transplant models (Kirk et al, Phil Trans R Soc Lond B, 2001, vol 356, pp. 691-702). However, the exact mechanism through which this blockade can inhibit rejection of transplanted tissue is still not clear. As described in Kirk et al, CD40-CD154 interactions may amplify the immune response. Thus, clearly blocking these interactions would be beneficial.

Furthermore, a remarkable spectrum of chronic inflammatory conditions can be blocked or substantially reduced by disrupting the CD40-CD154 system. These studies typically employ either mice with targeted disruption of either CD40 or CD154 genes, or use neutralizing monoclonal anti-CD154 antibodies. These antibodies appear to work by disrupting the communication bridge constructed by CD40-CD154. The animals in these experimental models appear to be no worse for having this system disrupted for months.

At least two different companies are testing anti-human CD154 antibodies for efficacy in diseases such as systemic lupus erythematosus, graft-versus-host disease, and tissue transplantation. Trials are ongoing with much promise for success. As these trials proceed, the utility of disrupting the CD40-CD154 system in human disease will become clear.

The fact that monoclonal antibody disruption of the CD40-CD154 pathway works well for blunting acute and chronic inflammation suggests that this and other options for blocking this pathway hold promise as therapeutic agents. In addition, recent reports that agonistic anti-CD40 antibodies can also reduce progression and severity of a murine model for rheumatoid arthritis, suggest that activating agents of this pathway may also be used in therapy of pathological cases of chronic inflammation.

A critical role for CD40-CD154 has been established for several autoimmune diseases, including lupus nephritis, systemic lupus erythematosus, rheumatoid arthritis, and multiple sclerosis Treatment of such diseases by blocking the costimulatory pathway involving CD40-CD154 are currently being tested (Kyburz D, Carson D A, Corr M., Arthritis Rheum. 2000 November; 43(11):2571-7; Kelsoe G., J Clin Invest. 2003 November; 112 (10):1480-2; Huang W X, Huang P, Hillert J. Mult Scler. 2000 April; 6(2):61-5). Studies using several animal models of autoimmune diseases show that disease symptoms can be blocked or substantially reduced by disrupting the CD40-CD154 system. Particularly encouraging are the reports showing that concurrent therapy with anti-CD154 and CTLA4-Ig (a soluble fusion protein between an homologue of the costimulatory molecule CD28 and the Fc portion of IgG 1) had dramatic synergistic effects that not only block disease and inhibit autoantibody production, but also prevent clonal expansion of autoreactive T-cells, emphasizing the potential value of combining agents that target distinct molecular pathways in immune-mediated diseases.

The involvement of CD40-CD154 in lupus, nephritis and SLE has been extensively investigated. Several models of murine lupus have been used to investigate the potential therapeutic efficacy of interrupting the CD40-CD154 system, and all have shown impressive inhibition of autoantibody production and nephritis, and improved survival. Concurrent therapy with anti-CD154 antibodies and CTLA4-Ig showed dramatic synergistic effects that lasted long after treatment was discontinued. Particularly encouraging are the findings that treated mice were shown to maintain the capacity to mount an effective immune response after completion of therapy.

Phase I clinical trials with anti-CD154 antibodies were carried out in patients with SLE (Kalunian K C., et al. Arthritis Rheum. 2002 December; 46(12):3251-8). These studies indicated that the agent was well tolerated. However, in another study, thromboembolic complications were reported, possibly due to the particular antibody that was used (Koyama I, et al., Transplantation. 2004 Feb. 15; 77(3):460-2; Kawai T, et al., Nat Med. 2000 February; 6(2):114). Some anti-CD40 antibodies are known to be stimulatory, for example, acting as agonists rather than antagonists. Thus, the precise nature of the antibody being used would be expected to result in the varying appearance of many effects related to CD40-CD154 interactions, in addition to unexpected effects that are not related to these interactions. The synovial tissue in RA patients is enriched with mature antigen presenting cells (APCs) and many lymphocytes. Interactions and signaling through the costimulatory CD40-CD154 and CD28-CD80/86 molecules are involved in the initiation and amplification of the inflammatory reactions in the synovium. Thus, blocking such signaling pathways might provide a specific immunotherapeutic approach for the treatment of RA. Indeed, prevention of collagen-induced arthritis (CIA), a murine model for RA, was observed upon administration of anti-CD154 antibody. Treatment with anti-CD154 also prevented arthritis development in a model of immunoglobulin-mediated arthritis.

CD40-CD154 interactions play a critical role in T cell priming, and are involved in tolerance induction (Quezada S A, Jarvinen L Z, Lind E F, Noelle R J. Annu Rev Immunol. 2004; 22:307-28; Vermeiren J, et al, Clin Exp Immunol. 2004 February; 135(2):253-8; Graca L, et al, Immunol Res. 2003; 28(3):181-91). Ample experimental evidence demonstrates that anti-CD154 antibodies are potent inhibitors of allograft rejection in many diverse transplant models. The efficacy of anti-CD154 therapy in rodent allografts, such as skin, cardiac, islet and bone marrow, all showed that a brief course of therapy at the time of transplantation led to prolonged or indefinite allograft survival. Treatment with CTLA-Ig was synergistic with anti-CD154 therapy. In non-human primates, treatment with anti-CD154 has been remarkably successful in preventing acute renal allograft rejection. In this system, anti-CD154 appears capable of preventing allograft rejection and establishing a long lasting state of donor-specific hyporesponsiveness that is not dependent on continuous immunosuppressive medication. Anti-CD154 therapy was also shown to prevent islet cell rejection and prolong cardiac allograft survival in non-human primates. The durability of anti-CD154 therapy was very impressive when compared with conventional immunosuppression.

Allogeneic bone marrow transplantation is frequently performed for the treatment of haematological malignancies and aplastic anaemia. However, graft-versus-host disease (GVHD) is still the major complication of this procedure, resulting in immune deficiency, infection, organ damage and leading occasionally to patient death. Blocking strategies of co-stimulatory signals, including CD40-CD154, are being evaluated as targets of therapeutic intervention for GVHD (Pan Y, et al, Transplantation. 2003 July 15; 76(1):216-24; Appleman L J, et al, Leuk Lymphoma. 2002 June; 43(6):1159-67). Treatment with sublethal radiation and anti-CD154 antibody prevented GVHD in mice receiving allogeneic bone marrow cells. These mice accepted donor-origin, but not third party skin allografts. An ex-vivo approach has been described, in which the blockade of the CD40-CD154 interactions by anti-CD154 induces donor bone marrow cells to become tolerant to host alloantigens, and prevents GVHD in mice. In addition, a similar approach led to donor-specific tolerance to secondary skin grafts.

Atherosclerosis is a leading cause of cardiovascular disease, and the most prevalent cause of death in the western world. Recently, atherosclerosis has been associated with chronic inflammation, linking it to the immune system (Urbich C, Dimmeler S., Can J Cardiol. 2004 May; 20(7):681-3). The presence of CD154 on platelets and the known ability of platelet-bound CD154 to activate endothelial cells, suggest that a critical role may be to initiate chemotactic and adhesion signals at the site of vascular trauma. An emerging body of evidence supports a key role for the CD40-CD154 system in atheroma progression.

Recent data from experimental animal models of atherosclerosis show that disruption of the CD40-CD154 pathway can prevent atherosclerotic progression and may reverse established lesions. Blockade of this pathway by this and other biological molecules may prove valuable in the treatment of atherosclerosis (Phipps R P, Curr Opin Investig Drugs. 2001 June; 2(6):773-7). Clinical trials are being currently conducted to ascertain the utility of disrupting CD40-CD154 interactions in human disease (Xu Y, Song G., J Biomed Sci. 2004 July-August; 11 (4):426-38).

In most organs, tissue injury is followed by cycles of inflammation and repair. When injury is repetitive or larger in magnitude, this frequently results in scarring or fibrosis. Fibrogenic pathologies are a characteristic feature of a wide spectrum of diseases in many organ systems. Tissue fibrosis can lead to significant organ dysfunction and resulting patient mortality.

There is increasing evidence that generation of specific cytokine patterns by immune and structural cells, and interactions between these cells via the CD40-CD154 pathway, may mediate many of the key events involved in fibrogenesis (Kaufman J, Sime P J, Phipps R P., J. Immunol. 2004 Feb. 1; 172(3):1862-71; Kaufman J et al, Chest. 2001 July; 120(1 Suppl):53S-55S). Following acute injury, infiltrating platelets and inflammatory cells can both activate a variety of local structural cells, including fibroblasts, through the CD40-CD154 system. This interaction triggers production of proinflammatory cytokines, expression of cell adhesion molecules, and induction of cyclooxygenase 2 (COX-2), leading to a pro-fibrogenic response. Thus, interruption of the CD40-CD154 system in acute injury, might reduce inflammation and avoid progression to end-stage fibrosis. Indeed, use of anti-CD154 was effective in protecting against injury and fibrosis in two mouse models: hyperoxic lung injury and radiation-induced lung injury (Adawi A, et al, Clin Immunol Immunopathol. 1998 December; 89(3):222-30; Adawi A, et al, Am J Pathol. 1998 March; 152(3):651-7).

CD40 upregulation is involved in pathogenic cytokine production in patients with inflammatory bowel diseases (IBD) (Danese S, et al, Gut. 2003 October; 52(10):1435-41). Increased expression of CD40 in B-lymphocytes, monocytes and dendritic cells is observed in patients with ulcerative colitis and Crohn's disease. Expression of CD40 and CD154 in B cells/macrophages and CD4+ T cells, respectively, was significantly increased in inflamed mucosa from these patients. Blocking the CD40-CD154 pathway with anti-CD154 antibody in a chronic murine colitis model ameliorates symptoms even after onset of disease. Thus, blockade of CD40-CD154 interactions may have therapeutic effects for IBD patients (Liu Z, et al, J. Immunol. 2000 Jun. 1; 164(11):6005-14).

The CD40-CD154 system plays a critical role in the response of the immune system to an invading pathogen, leading to an antigen-driven lymphoproliferative process. When downregulation of this tightly controlled mechanism is impaired, lymphoproliferative disorders may occur. CD40 expression is elevated in malignant B- and T-cell lymphomas, and in Reed-Sternberg cells of Hodgkin's disease. CD154 is constitutively expressed in several types of B-cell lymphoid malignancies. Furthermore, approximately 50% of patients with these malignancies have elevated levels of biologically active soluble CD154 in their serum. The effect of CD40 activation in B-cell malignancies has been examined extensively by use of activating anti-CD40 antibodies or soluble CD154. Whenever primary human malignant B cells were analyzed, CD40 activation consistently enhanced malignant cell survival and mediated their resistance to chemotherapy (Ottaiano A, et al, Tumori. 2002 September-October; 88(5):361-6; Fiumara P, Younes A., Br J Haematol. 2001 May; 113 (2):265-74; Kipps T J, Chu P, Wierda W G. Semin Oncol. 2000 December; 27(6 Suppl 12):104-9; Szocinski J L., et al. Blood. 2002 July 1; 100(1):217-23).

Taken together, the co-expression of CD40 and CD154 by malignant B cells, the presence of soluble CD154 in the sera of these patients, and the ability of CD40 activation to enhance malignant B-cell survival, suggest that CD40/CD154 may provide an autocrine/paracrine survival loop for malignant B cells. Thus, interrupting CD40/CD154 interaction may be of therapeutic value in patients with B-cell lymphoid malignancies. Anti-CD154, but surprisingly also stimulatory antibodies to CD40, were successfully tested as immunotherapy for malignant B cell tumors in murine models.

Elevated expression of CD40 was described in other forms of cancer, including epithelial neoplasia, nasopharyngeal carcinoma, osteosarcoma, neuroblastoma and bladder carcinoma. Recombinant soluble CD154 inhibited the growth of CD40(+) human breast cell lines in vitro, due to increased apoptosis. In addition, treatment of tumor-bearing mice with this molecule resulted in increased survival.

Another aspect of CD40/CD154 in the treatment of malignancies is the potential use of CD154 in immune gene therapy, since CD40/CD154 interaction has been shown to be critical for generating protective T cell-mediated anti-tumor response (Tong A W, Stone M J., Cancer Gene Ther. 2003 January; 10(1): 1-13; Kipps T J, Int J Hematol. 2002 August; 76 Suppl 1:269-73). In this approach, CD154 is transferred ex-vivo into neoplastic cells, by infection with a modified adenovirus. The results of a Phase I study in CLL patients show induction of autologous cytotoxic T cells capable of destroying the neoplastic B cells, concomitant with significant reduction in leukemic cell counts and lymph node sizes. Furthermore, this approach appears to enhance antibody-dependent cellular cytotoxicity, and thereby augment the activity of antitumor monoclonal antibody therapy. Thus, this approach alone or in combination with tumor-specific Mab therapy (such as Rituxan, anti-CD20), may offer an effective strategy for the treatment of B-cell malignancies. Transduction of tumor cells ex vivo with CD154, in solid tumors such as neuroblastoma and squamous cell carcinoma, can induce immune responses against the tumor cells, mediating rejection or impeding tumor growth.

Activated T-lymphocytes not only express cell membrane-associated but also soluble CD154. The kinetics of soluble CD154 (sCD154) expression resemble expression patterns observed for the membrane-associated form, though the mechanisms of generation and/or release of sCD154 remain poorly understood. Several studies suggest that sCD154 retains the ability to interact with CD40. Recently, the soluble forms of CD154 have received more attention, particularly in association with certain human diseases. Enhanced levels of sCD154 have been detected in patients with disorders such as active SLE, unstable angina, and B-Cell lymphoma (Komura K et al, J Rheumatol. 2004 March; 31(3):514-9; Conde I D, Kleiman N S., N Engl J. Med. 2003 Jun. 19; 348(25):2575-7; author reply 2575-7, Heeschen C, et al, N Engl J. Med. 2003 Mar. 20; 348(12):1104-11.

Soluble CD40 (sCD40) was detected in culture supernatants from CD40-positive cell lines, but not from CD40-negative cells. A substantial proportion of sCD40 in these cultures retained ligand binding activity. High levels of sCD40 were also observed in supernatants from AIDS-related lymphoma B-cell lines. sCD40 that was expressed by B cells was shown to bind CD154 on activated T cells, and is thought to regulate CD40-CD154 in a negative fashion. sCD40 was also detected in serum and urine of healthy donors, and was highly elevated in patients with impaired renal function, including chronic renal failure, haemodialysis and chronic ambulatory peritoneal dialysis (CAPD) patients (Contin C, Immunology. 2003 September; 110(1): 131-40). Patients with neoplastic disease and chronic inflammatory bowel disease (CIBD) (Schwabe, R. F., et al, Clin. Exp. Immunol, 1999, 117:153-158) showed slight but significant elevations of sCD40 in their serum.

A recent study suggested that sCD40 can be created through alternative splicing (Tone, M., et al., 2001, PNAS 98:1751-1756). As such, sCD40 molecules may have unique antigenic epitopes, distinct from CD40, which could be used to raise sCD40-specific antibodies.

At least one study suggests that expression of sCD40 regulates CD40-CD154 interactions in a positive fashion. Given the ample evidence for a critical role of CD40-CD154 in injury, inflammation and cancer, it appears that targeting this system may prove to play an important therapeutic role in abating inflammation in a variety of diseases and in cancer treatment. Blocking the CD40-CD154 system could be performed by using molecules that act as CD40 antagonists, or that disrupt CD40-CD154 interactions. Reports that agonistic anti-CD40 antibodies can also reduce severity of disease and disease progression suggest that activating this pathway may be useful for therapy of pathological cases of chronic inflammation.

Proteins that are able to bind to or otherwise interact with CD40 could be useful for manipulation of various cellular functions, including, for example, humoral immune responses and apoptosis. Such an effective agent, whether an agonist or antagonist, can modulate cell functions such as humoral immune responses or cell growth. These agents can also be used to modulate cell proliferation, immune response, apoptosis etc., for example for treating cancer or autoimmune diseases.

Monoclonal antibody targeting of the CD40-CD154 pathway has shown beneficial effects in a number of experimental animal models. However, whether these techniques can be applied to humans remains to be determined, since treatment with humanized antibodies has obvious limitations. Other options for modulating this pathway with higher specificity and efficacy, such as sCD40, hold promise as therapeutic agents.

Splice variants of the transcript that encodes CD40 have been isolated, characterized and cloned. These splice variants include naturally occurring sequences obtained by alternative splicing of the known wild type CD40 gene depicted as CD40 HUMAN Swiss Prot. under Accession Number P25942, which is incorporated herein by reference. These splice variants are not merely truncated forms, or fragments of the known gene, but rather novel sequences that naturally occur within the body of individuals. Different splice variants encoded by a single gene may be expressed in vivo in different physiological situations and may result in activation of distinct cellular pathways. These splice variants include nucleic acid molecules that encode the extracellular region of CD40 or a fragment thereof, linked to a unique tail sequence. The extracellular region may be fully conserved, or there may be deletions, insertions or substitutions. In some variants the translation product of the splice variant is a soluble protein that retains the CD40 function of binding to CD40 ligands such as CD154 or CD40 itself.

### SUMMARY OF THE INVENTION

The present invention provides a specific CD40 receptor splice variant, termed “skipping 6”, which has unique pharmaceutical properties, including a specific binding profile for binding to the CD154 ligand. This CD40 variant can compete with the membrane CD40 for binding to CD154 ligand. Binding to the CD154 ligand disrupts the CD154-CD40 receptor interaction and down regulates the activity of the pathway.

Interruption of this interaction, and hence down regulation of pathway activity, is useful for treating diseases where it is desired to decrease the activity of the immune system. A decrease in the activity of the immune system is desired in, e.g., treating autoimmune diseases, chronic inflammatory diseases or in inhibiting rejection of implanted cells or tissue.

The skipping 6 mRNA transcript is shown hereby to have a physiological expression pattern that is different from that of the wild type or previously known CD40 receptor (termed herein “CD40 wild type” or “CD40WT”). Namely, the level of the skipping 6 transcript rises when apoptosis is induced in erythroleukemic cells, while the level of the wild type CD 40 receptor decreases when apoptosis is induced. The diseases involving apoptosis can be divided into two groups: those in which there is an increase in cell survival (or disease associated with inhibition of apoptosis), and those in which there is an increase in cell death (and hence hyperactive apoptosis). There are many studies demonstrating that cell apoptosis plays a relevant role in the etiology of many diseases, and that a wide range of pharmacologic agents (cytotoxic agents, hormones, anti-inflammatory drugs) are effective through inducing apoptosis of target cells (see for example Ramirez et al. 1999. Apoptosis and disease. Alergol. Immunol. Clin. 6: 367-374).

As is described below, skipping 6 CD40 variant has also been found to lower the level of the cytokine RANTES when administered to a mixture of human peritoneal cells and mouse fibroblasts transfected to express the CD154 ligand, as compared to when an interferon control was administered alone. RANTES is a cytokine indicative of T cell activation.

The skipping 6 protein is encoded for by SEQ ID NO: 1, shown in the attached sequence listing. The availability of the naturally occurring protein of the present invention, as an alternative to other known artificial agents which can interrupt the naturally occurring CD40 receptor CD40-ligand interaction, provides therapeutic alternatives to those patients who do not respond to commercially available CD40 receptor blocking agents, or as an alternative to CD40 receptor blocking agents which can cause substantial adverse side effects.

The present invention thus provides, in a preferred embodiment, a pharmaceutical composition comprising a pharmaceutically acceptable carrier, and as an active ingredient an agent comprising a protein having an amino acid sequence selected from:
i. the amino acid sequence depicted in SEQ. ID No. 1;
ii. a fragment of at least 10 amino acids the amino acid sequence of (i), having at least four consecutive amino acids of the segment 166-203 of SEQ. ID No. 1, said fragment still having CD40-L binding properties substantially as those of the sequence of (i);
iii. a variant of the amino acid sequence of (i) or (ii) wherein up to about 20% of the amino acids have been replaced, chemically modified or deleted, and/or having at least about 70% homology, optionally at least about 80% homology, preferably at least about 85% homology, more preferably at least about 90% homology and most preferably at least about 95% homology to the amino acid sequence of (i) or (ii) wherein the sequence substantially maintains the CD40-L binding properties of (i);
iv. a chimeric protein comprising the amino acid sequence of (i), (ii) or (iii), conjugated to another entity.
   v. An isolated chimeric polypeptide encoding for CD40-skipping 6, comprising a first amino acid sequence being at least 90% homologous to MVRLPLQCVLWGCLLTAVHPEPPTACREKQYLINSQCCSLCQPGQKLVSDCT EFTETECLPCGESEFLDTWNRETHCHQHKYCDPNLGLRVQQKGTSETDTICTC EEGWHCTSEACESCVLHRSCSPGFGVKQIATGVSDTICEPCPVGFFSNVSSAFE KCHPWT corresponding to amino acids 1-165 of TNR5_HUMAN (SEQ ID NO:23), which also corresponds to amino acids 1-165 of CD40-skipping 6 (SEQ ID NO:1), and a second amino acid sequence being at least 70%, optionally at least 80%, preferably at least 85%, more preferably at least 90% and most preferably at least 95% homologous to a polypeptide having the sequence RSPGSAESPGGDPHHLRDPVCHPLGAGLYQKGGQEANQ corresponding to amino acids 166-203 of CD40-skipping 6 (SEQ ID NO:24), wherein said first amino acid sequence and second amino acid sequence are contiguous and in a sequential order.
   vi. An isolated polypeptide encoding for a tail of CD40-skipping 6, comprising a polypeptide being at least 70%, optionally at least about 80%, preferably at least about 85%, more preferably at least about 90% and most preferably at least about 95% homologous to the sequence RSPGSAESPGGDPHHLRDPVCHPLGAGLYQKGGQEANQ in CD40-skipping 6.

Reference is further made to the nucleic acid sequence of SEQ ID No 2, which is an exemplary nucleic acid sequence coding for SEQ ID No. 1, which may be used in the production of SEQ ID No. 1, and/or may be used as a probe and/or to design such a probe in the detection of expression of the protein in a sample.

The phrase “substantially maintains the CD40-L binding properties” means the protein, e.g., a CD40-skipping 6 protein, variant or fragment thereof, binds specifically to a CD40-L. In some embodiments, the protein binds to a CD40L molecule at a concentration at which binding of wild-type CD40 is not detected. In the following the term “skipping 6” refers in general to any one of the sequences (i)-(iv) above, all of which are characterized in having at least part of the unique tail of amino acids 166-203 at SEQ ID NO:1, and preferably are unique, as compared to soluble CD40 and other splice variants of CD40 in that they lack the exon 6 of the extracellular domain.

According to certain embodiments of the invention, in (ii), the CD40-L binding properties are due to the presence of particular (or at least similar) amino acids at positions E74, Y82, N86, D84, E114, E117 of the CD40 (for both the known and variant proteins), so that the fragment of (ii) above preferably comprises one or more regions containing these amino acids believed to be critical for binding CD40 binding domains linked to each other (either in the order appearing in the native protein or in another order) optionally through spacers, and further linked to said at least four consecutive amino acids of amino acids 166-203 at SEQ ID NO:1. The amino acids at positions E74, Y82, N86, D84, E114, E117 of the CD40 protein are preferably either maintained as in the parent sequence, or substituted by conservative substitution.

The segment comprising amino acids “166-203” of SEQ ID NO: 1 is the unique tail of the CD40 splice variant skipping 6, a segment which does not appear in the wild type CD40 sequence or in any other of the known splice variants.

Four consecutive amino acids may be any four amino acids of this tail such as, for example, amino acids at positions 166-169, 167-170, 168-171 . . . 200-203 of the CD40 skipping 6 variant, SEQ ID NO:1.

The consecutive amino acids may be five (for example, amino acids at positions 166-170, 167-171 . . . 199-203 of the CD40 skipping 6 variant, SEQ ID NO:1), six (for example, amino acids at positions 166-171 . . . 198-203 of the CD40 skipping 6 variant, SEQ ID NO:1), seven, eight or nine.

Preferably at least 10 consecutive amino acids of the unique segment comprising amino acids 166-203 of SEQ ID NO:1, and more preferably all of the amino acids of this unique segment, are present in the fragment.

The term “up to about 20%” means that at least about 80% of the variant sequence are identical to those of (i) or (ii), so that a combination of preferably no more than about 20 amino acids has been deleted, and/or replaced and/or chemically modified.

Moreover, in an embodiment of the invention, preferably up to about 15% of the amino acids have been replaced, chemically modified or deleted (i.e. about 85% are identical with (i) or (ii), wherein the sequence maintains the CD40-L binding properties of (i). In another embodiment, preferably up to about 10% of the amino acids have been replaced, chemically modified or deleted (i.e. about 90% are identical with (i) or (ii), wherein the sequence maintains the CD40-L binding properties of (i). In yet another embodiment, preferably up to about 5% of the amino acids have been replaced, chemically modified or deleted (i.e. about 95% are identical with (i) or (ii), wherein the sequence maintains the CD40-L binding properties of (i).

Additionally, in certain embodiments of the invention, in the chimeric protein of (IV, which is a previously described chimeric protein comprising the amino acid sequence of (i), (ii) or (iii), conjugated to another entity), the amino acid sequence of (i), (ii) or (iii) is preferably conjugated to an entity selected from a member of the following group: an antibody or an antibody fragment, preferably an F_{c} fragment, a glycoprotein, a fragment of the comp protein (pentameric scaffold derived from the coiled-coil domain of cartilage oligomeric matrix protein (COMP); Holler N et al, J Immunol Methods. 2000 April 3; 237(1-2):159-73), b-zip (Morris A. E., et al, JBC, 274: 418-423, 1999). Optionally, in some embodiments the antibody fragment is obtained from or is a derivative or fragment of the Fc region of an antibody of IgG1.

Further, in some embodiments, in (i), (ii), (iii) or (iv), up to about 20% of the amino acid of the native sequence has been replaced with a naturally or non-naturally occurring amino acid or with a peptidomimetic organic moiety; and/or up to about 20% of the amino acids have their side chains chemically modified and/or up to about 20% of the amino acids have been deleted, provided that at least about 80% of the amino acids in the original variant sequence of (i), (ii), (iii) or (iv) are maintained unaltered, and provided that the amino acid maintains the biological activity of the original variant sequence of (i), (ii), (iii) or (iv).

Still further, in some embodiments, in (i), (ii), (iii), or (iv), at least one of the amino acids is replaced by the corresponding D-amino acid, which replacement increases the protein's resistance to degradation by naturally present enzymes.

Additionally, in certain embodiments, in (i), (ii), (iii), or (ic) the peptidic backbone of at least one of the amino acids is altered to a non-naturally occurring peptidic backbone, which replacement increases the protein's resistance to degradation by naturally present enzymes.

According to another embodiment of the present invention, there is provided a method for treatment of a disease, wherein a beneficial therapeutic effect is achieved by the interruption of the CD40-R-CD40-L interaction, comprising administering, to an individual in need of such treatment, a therapeutically effective amount of the composition of the present invention. This composition comprises a pharmaceutically acceptable carrier, and as an active ingredient an agent comprising an amino acid sequence selected from:
(i) the amino acid sequence depicted in SEQ. ID No. 1;
(ii) a fragment of at least 10 amino acids of the amino acid sequence of (i), having at least four consecutive amino acids of the segment 166-203 of SEQ. ID No. 1, said fragment still having CD40-L binding properties substantially as those of the sequence of (i);
(iii) a variant of the amino acid sequence of (i) or (ii) wherein up to about 20% of the amino acids have been replaced, chemically modified or deleted, and/or having at least about 70% homology, optionally at least about 80% homology, preferably at least about 85% homology, more preferably at least about 90% homology and most preferably at least about 95% homology to the amino acid sequence of (i) or (ii), wherein the sequence substantially maintains the CD40-L binding properties of (i);
(iv) a chimeric protein comprising the amino acid sequence of (i), (ii) or (iii), conjugated to another entity.

Preferably, the disease is selected from graft-versus-host disease, transplant rejection, atherosclerosis, pulmonary fibrosis, autoimmune diseases such as: lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, asthma, cryoglobulinemia, primary biliary sclerosis and pernicious anemia; end-stage fibrosis, hyperoxic injuries, radiation-induced injuries, cancers (human leukemias, lymphomas, and multiple myeloma) epithelial neoplasia, nasopharyngeal carcinoma, osteosarcoma, neuroblastoma and bladder carcinoma, AIDS-related lymphoma impaired renal function, including chronic renal failure, hemodialysis and chronic ambulatory peritoneal dialysis (CAPD) patients.

Most preferably, the disease is selected from: graft vs. host disease (actual diseases for which experiments are performed, e.g. the vivo model currently being developed, which will be included in the Examples section) and transplant rejection.

Further, the present invention relates to use of a sequence as defined in (i)-(iv) above, for preparing a medicament for the treatment of a disease, wherein a beneficial therapeutic effect is achieved by the interruption of the CD40-R-CD40-L interaction, wherein such a disease may be termed CD40-related disease.

Moreover, in such use, the disease can be selected from graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, asthma, cryoglobulinemia, primary biliary sclerosis and pernicious anemia, end-stage fibrosis, hyperoxic injuries, radiation-induced injuries, cancers, epithelial neoplasia, nasopharyngeal carcinoma, osteosarcoma, neuroblastoma and bladder carcinoma, AIDS-related lymphoma, impaired renal function, including chronic renal failure, haemodialysis and chronic ambulatory peritoneal dialysis (CAPD) patients.

Additionally, most preferably during use, the disease is selected from Atherosclerosis, Chronic Inflammatory Disease, Cancer and Immune Rejection of Transplanted Organs (actual diseases for which experiments are included and mentioned in Examples).

The present invention is further based on the surprising finding that a combination of the “skipping 6” protein with an antibody selective to skipping 6 (i.e. which binds to skipping 6 but does not bind with soluble CD40) has better CD40-CD40L interrupting properties than the skipping 6 variant alone.

This surprising finding is evidenced by the fact that the ED₅₀ without the antibody of skipping 6 was about 100 nM, while with the antibody was about 2 nM—about 50 times less.

Thus in accordance with the present invention a pharmaceutical composition comprising both the skipping 6 protein (as defined herein) and the selective antibody to the skipping 6 protein is preferred.

Furthermore, as the splice variant skipping 6 is naturally expressed in the body, at least a certain level of the skipping 6 protein may be expected a priori to be present in the circulation. Without wishing to be limited by a single hypothesis, administration of the skipping 6 selective antibody itself may, together with the basal levels of naturally present skipping 6 protein, cause interruption of the CD40-CD40L interaction.

Thus the present invention concerns a pharmaceutical composition comprising a pharmaceutically acceptable carrier and as an active ingredient an antibody capable of selectively binding to the amino acid of SEQ ID NO: 1, while essentially not binding to wild-type soluble CD40.

As used herein the term “unique tail” is meant to refer to the amino acid sequence at the C terminus of the CD40 splice variant, which sequence does not appear in the wild type CD40. The unique tail region of the CD40 splice variant SEQ ID NO:1 spans amino acids 166-203 of SEQ. ID No. 1.

In the present invention, the term “ligand” or “CD40 ligand” or “CD40-L” is meant to refer not only to CD154, but to any other compounds such as TRAF3 or TRAF2 which are known to interact with CD40.

In the present invention, the term “CD40-R-CD40-L interaction” refers to the interaction between the CD40 receptor and at least one of its ligands.

As used herein, the term “fragments” as applied to protein fragments of the CD40 splice variant refers to those fragments which are at least 10 amino acids long which include at least 4 consecutive amino acids of the unique tail region (defined as amino acids 166-203 in SEQ ID NO:1). In some preferred embodiments the fragments includes 5, 6, 7, 8, 9, preferably above 10, most preferably all the amino acids of the unique tail region. In some preferred embodiments of SEQ ID NO:1, the fragment includes 10, 11, 12, 13, 14, 15, 16, or 17 amino acids of the unique tail region.

It should be noted that in accordance with the invention several fragments which in the native skipping 6 variant of SEQ ID NO: 1 are separated may be linked to each other in tandem either directly or through suitable spacers.

As used herein the term “nucleic acid sequence” is meant to refer to a sequence composed of DNA nucleotides, RNA nucleotides or a combination of both types and may include natural nucleotides, chemically modified nucleotides and synthetic nucleotides.

The term “amino acid” refers either to one of the 20 naturally occurring amino acids, to a peptidomimetic thereof (see below), or to a D or L residue having the following formula:
—NH—CHR—CO—
wherein R is an aliphatic group, a substituted aliphatic group, a benzyl group, a substituted benzyl group, an aromatic group or a substituted aromatic group and wherein R does not correspond to the side chain of a naturally occurring amino acid. This term also refers to the D-amino acid counterpart of naturally occurring amino acids. Amino acid analogs are well known in the art; a large number of these analogs are commercially available. Without wishing to be limited by a single hypothesis, many times the use of non-naturally occurring amino acids in a peptide has an advantage, in that the peptide is more resistant to degradation by enzymes which fail to recognize these amino acids.

As used herein the term “substitution” refers both to conservative and non conservative substitutions.

The term “conservative substitution” in the context of the present invention refers to the replacement of an amino acid present in the native sequence, with a naturally or non-naturally occurring amino or a peptidomimetic thereof (see below) having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

To produce conservative substitutions by non-naturally occurring amino acids it is also possible to use amino acid analogs (synthetic amino acids) well known in the art. A peptidomimetic of the naturally occurring amino acid is well documented in the literature and known to the skilled practitioner.

When effecting conservative substitutions the substituting amino acid should have the same or a similar functional group in the side chain as the original amino acid.

The following are some non-limiting examples of groups of naturally occurring amino acids or of amino acid analogs. Replacement of one member in the group by another member of the group will be considered herein as a conservative substitution:

Group I includes: leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes: glycine, alanine, valine, serine, cysteine, threonine and a modified amino acid having an ethyl side chain. Preferably Group II includes glycine and alanine.

Group III includes: phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes: glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)_{3—}COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes: histidine, lysine, arginine, N-nitroarginine, β-cycloarginine, μ-hydroxyarginine, N-amidinocitruline and 2-amino-4-guanidinobutanoic acid, homologs of lysine, homologs of arginine and ornithine. Preferably, Group V includes histidine, lysine, arginine, and ornithine. A homolog of an amino acid includes from 1 to about 3 additional methylene units in the side chain.

Group VI includes: serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

The term “non-conservative substitutions” concerns replacement of the amino acid as present in the skipping 6 protein sequence of SEQ ID NO:1 by another naturally or non-naturally occurring amino acid, having different electrochemical and/or steric properties, for example as determined by the fact the new replacement amino acid is not in the same group classification as the replaced amino acid of the native protein sequence. Those non-conservative substitutions which fall under the scope of the present invention are those which still constitute a compound having CD40-L binding properties. Because D-amino acids have hydrogen at a position identical to the glycine hydrogen side chain, D-amino acids or their analogs can often be substituted for glycine residues, and are a preferred non-conservative substitution

A “non-conservative substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid.

Alternatively, a functional group may be added to the side chain, deleted from the side chain or exchanged with another functional group. Examples of non-conservative substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

As used herein the term “chemically modified”, when referring to a protein of the invention, is meant to refer to a protein where at least one of its amino acid residues is modified either by natural processes, such as processing or other post-translational modifications, or by chemical modification techniques which are well known in the art. Among the numerous known modifications typical, but not exclusive examples, include: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

As used herein the term “having at least 80% identity” with respect to two amino acid or nucleic acid sequence sequences, is meant to refer to the percentage of residues that are identical in the two sequences when the sequences are optimally aligned. Thus, 80% amino acid sequence identity means that 80% of the amino acids in two or more optimally aligned polypeptide sequences are identical.

As used herein the term “deletion” is meant to refer to the absence of one or more amino acids which may be at terminal or non terminal regions and which absence may be of several consecutive or non consecutive amino acid residues.

As used herein the terms “insertion” and “addition” is meant to refer to a change in a nucleotide or amino acid sequence which has resulted in the addition of one or more nucleotides or amino acid residues, respectively, as compared to the original sequence.

As used herein the term “substitution” is meant to refer to replacement of one or more amino acids by different amino acids. With regard to amino acid sequences, the substitution may be conservative or non-conservative.

As used herein the term “alternative splicing” is meant to refer to exon exclusion, deletion of terminal or non terminal sequences in the variants as compared to the original sequence, as well as to intron inclusion of sequences originally not appearing in the parent (wild type) sequence.

As used herein, the term “effective amount” refers to an amount of active ingredient which is an ingredient comprising any of the sequences (i) to (iv) or in order to prevent, ameliorate or cure a disease or postpone deterioration of a disease and is determined by such considerations as may be known in the art. The amount must be effective to achieve the desired therapeutic effect by administering the amino acid sequences of the invention, to a person in need thereof. The amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount.

According to preferred embodiments of the present invention, preferably any of the nucleic acid and/or amino acid sequences featured herein further comprises any sequence having at least about 70%, preferably at least about 80%, more preferably at least about 90%, most preferably at least about 95% homology thereto.

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all specific regions described hereni, optionally including other antibody epitopes as described herein) and/or polypeptides (including for all proteins). It should be noted that oligonucleotide and polynucleotide, or peptide and polypeptide, may optionally be used interchangeably.

According to preferred embodiments of the present invention, there is provided an antibody capable of specifically binding to an epitope of an amino acid sequence as described herein.

Optionally amino acid sequence corresponds to a tail as described herein. Also optionally, the antibody is capable of differentiating between a splice variant having the epitope and a corresponding known protein, such as the known or WT (wild type) CD40 protein described herein.

According to preferred embodiments of the present invention, there is provided at least one primer pair capable of selectively hybridizing to a nucleic acid sequence as described herein. According to other preferred embodiments, there is provided at least one oligonucleotide capable of selectively hybridizing to a nucleic acid sequence as described herein.

According to preferred embodiments of the present invention, there is provided a nucleic acid construct comprising the isolated polynucleotide as described herein.

Optionally, the nucleic acid construct further comprises a promoter for regulating transcription of the isolated polynucleotide in sense or antisense orientation.

Optionally, the nucleic acid construct further comprises a positive and a negative selection marker for selecting for homologous recombination events.

According to preferred embodiments of the present invention, there is provided a host cell comprising the nucleic acid construct as described herein.

According to preferred embodiments of the present invention, there is provided an isolated polypeptide comprising an amino acid sequence at least 70% identical to a polypeptide as described herein, as determined using the LALIGN software of EMBnet Switzerland (http://www.ch.embnet.org/index.html) using default parameters or an active portion thereof.

According to preferred embodiments of the present invention, there is provided an oligonucleotide specifically hybridizable with a nucleic acid sequence encoding a polypeptide as described herein.

According to preferred embodiments of the present invention, there is provided a pharmaceutical composition comprising a therapeutically effective amount of a polypeptide as described herein and a pharmaceutically acceptable carrier or diluent.

According to preferred embodiments of the present invention, there is provided a method of treating CD40-related disease in a subject, the method comprising upregulating in the subject expression of a polypeptide as described herein, thereby treating the CD40-related disease in a subject. Optionally, upregulating expression of said polypeptide is effected by:
(i) administering said polypeptide to the subject; and/or
(ii) administering an expressible polynucleotide encoding said polypeptide to the subject.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×106 cpm 32P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×106 cpm 32P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include 3H, 14C, 32P, and 35S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Optionally and preferably, nucleic acid sequence homology (identity) is determined using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2—), *-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by 15 recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

It will be appreciated that the present methodology for treatment may be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (ie., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Molecular and Subcellular Biology 31:217-239.

Alternatively or additionally, upregulation may be effected by administering to the subject at least one polypeptide agent of the polypeptides of the present invention or an active portion thereof, as described hereinabove. However, since the bioavailability of large polypeptides is relatively small due to high degradation rate and low penetration rate, administration of polypeptides is preferably confined to small peptide fragments (e.g., about 100 amino acids).

An agent capable of upregulating a CD40 polypeptide may also be any compound which is capable of increasing the transcription and/or translation of an endogenous DNA or mRNA encoding the CD40 polypeptide and thus increasing endogenous CD40 activity.

An agent capable of upregulating a CD40 may also be an exogenous polypeptide including at least a functional portion (as described hereinabove) of the CD40.

Upregulation of CD40 can be also achieved by introducing at least one CD40 substrate. Non-limiting examples of such agents include HOXC10 (Gabellini D, et al., 2003; EMBO J. 22: 3715-24), human securin and cyclin B1 (Tang Z, et al., 2001; Mol. Biol. Cell. 12: 3839-51), cyclins A, geminin H, and Cut2p (Bastians H, et al., 1999; Mol. Biol. Cell. 10: 3927-3941).

It will be appreciated that upregulation of CD40 can be also effected by administration of CD40-expressing cells into the individual.

CD40-expressing cells can be any suitable cells, such as lung, ovary, bone marrow which are derived from the individual and are transfected ex vivo with an expression vector containing the polynucleotide designed to express CD40 as described hereinabove.

Administration of the CD40-expressing cells of the present invention can be effected using any suitable route such as intravenous, intra peritoneal, and intra ovary. According to presently preferred embodiments, the CD40-expressing cells of the present invention are introduced to the individual using intravenous and/or intra organ administrations.

CD40-expressing cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Downregulating Methods and Agents

Downregulation of CD40 can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of downregulating expression level and/or activity of CD40.

One example, of an agent capable of downregulating a CD40 polypeptide is an antibody or antibody fragment capable of specifically binding CD40. Preferably, the antibody specifically binds at least one epitope of a CD40 as described hereinabove.

An agent capable of downregulating a CD40 transcript is a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the CD40 transcript mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl, T. 2001, ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a CD40 transcript is a DNAzyme molecule capable of specifically cleaving an mRNA transcript or DNA sequence of the CD40. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther. www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a CD40 transcript can also be effected by using an antisense polynucleotide capable of specifically hybridizing with an mRNA transcript encoding the CD40.

Design of antisense molecules which can be used to efficiently downregulate a CD40 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a CD40 transcript is a ribozyme molecule capable of specifically cleaving an mRNA transcript encoding a CD40. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10: 163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent capable of downregulating CD40 would be any molecule which binds to and/or cleaves CD40. Such molecules can be CD40 antagonists, or CD40 inhibitory peptide.

It will be appreciated that a non-functional analogue of at least a catalytic or binding portion of CD40 can be also used as an agent which downregulates CD40.

Another agent which can be used along with the present invention to downregulate CD40 is a molecule which prevents CD40 activation or substrate binding.

Each of the upregulating or downregulating agents described hereinabove or the expression vector encoding CD40 can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that treatment of CD40 related disease according to the present invention may be combined with other treatment methods known in the art (i.e., combination therapy).

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF THE DRAWINGS

FIG. 1: presents the pTen21 plasmid map and multiple cloning site sequences.

FIG. 2: schematic presentation of the pTen21 vector digested with EcoRV and BglII and ligated to the amplified PCR fragment of CD40 wtEC, digested with the same enzymes.

FIG. 3: schematic presentation of the pTen21-CD40 wtEC clone 7 vector digested with StuI and BglII and ligated to the amplified PCR fragment of CD40-skipping 6, digested with the same enzymes.

FIG. 4: shows the Vector pTen21-CD40 wtEC (EC refers to the extracellular domain of CD40 WT) full length sequence. The primers are marked in bold and underlined, in bold italic is the CD40 wtEC coding sequence. Also shown are the BamHI-EcoRV and BglII sites. The −signal peptide-encoding sequence is presented in the rectangle.

FIG. 5: shows the Vector pTen21-CD40_Skip6 full length sequence. The primers are marked in bold and underlined, in bold italic is the CD40_Skip6 coding sequence. Also shown are BamHI-EcoRV and BglII sites. The signal peptide is presented in the rectangle.

FIG. 6: presents the sequence determined for Clone 7 as described in SEQ ID NO:9, featuring the BamHI-EcoRV and BglII sites marked in bold, encompassing the CD40 wtEC sequence.

FIG. 7: presents the sequence determined for Clone 5 as described in SEQ ID NO:14, featuring the BamHI-EcoRV and BglII sites marked in bold, encompassing the CD40_Skip6 sequence, which is shown in bold italic.

FIG. 8: shows the Vector pTen21-Fc full length sequence. The OQBT primers are marked in bold and underlined, the Fc sequence is colored in bold italics. Also shown is the polylinker

FIG. 9: shows the Fc sequence within the pTen21-Fc vector, as shown in SEQ ID NO: 16, featuring the XhoI and KpnI sites marked in bold, encompassing the Fc sequence, which is shown in bold italics.

FIG. 10: schematic presentation of the pTen21-Fc clone 19 digested with EcoRV and BglII and ligated to the amplified PCR fragment of CD40 wtEC, digested with the same enzymes.

FIG. 11: shows the Vector pTen21-cd40 wtEC-Fc full length sequence. The primers are marked in bold and underlined. The CD40 wtEC-Fc fusion sequences are shown in bold italics, and are separated by the tacgta sequence.

FIG. 12: presents the sequence determined for clone 37, as presented in SEQ ID NO: 19, featuring the BamHI-EcoRV and KpnI sites marked in bold. The CD40 wtEC-Fc fusion sequences are shown in bold italics, and are separated by the tacgta sequence.

FIG. 13: schematic presentation of the pTen21-CD40 wtEC-Fc clone 37 vector digested with StuI and BglII and ligated to the amplified PCR fragment of CD40-skipping 6, digested with the same enzymes.

FIG. 14: shows the Vector pTen21-CD40_Skip6-Fc vector, presented in SEQ ID NO: 21, where the primers are marked in bold and underlined. The CD40 wtEC-Fc fusion sequences are shown in bold italics, and are separated by the tacgta sequence. Also shown are the polylinker and the signal peptide-encoding sequence, which is shown with a rectangle.

FIG. 15: presents the sequence determined for clone 33, as presented in SEQ ID NO: 22, where the internal StuI, BamHI and BglII sites are underlined. The BamHI and EcoRV sites upstream of the ATG are in Bold. The CD40 wtEC-Fc fusion sequences are shown in bold italics, and are separated by the tacgta sequence.

FIG. 16: Western blot analysis of the purified CD40 proteins as follows: line 1 presents CD-40 wtEC-Fc protein, line 2 presents CD-40 wt-Fc protein, line 3 presents CD-40 skipping6-Fc protein, and line 4 presents the TNFRII-Fc negative control, recognised by a commercially available polyclonal antibody N-16 (polyclonal rabbit antibody from Santa Cruz Biotechnology, Inc. (Santa Cruz CA, USA; Cat num. Sc-974)).

FIG. 17: Binding profile of CD40-Skipping 6 protein to the immobilized CD154 ligand in ELISA plates.

FIGS. 18A-G: Results of FACS analysis, demonstrating sCD40 binding to CD154 ligand. FIGS. 18A-D represent CD-40-Fc binding, while FIGS. 18 E-G represent the binding of CD40 without the Fc. Detailed description of the experiments is provided in Example 4 below. FIG. 18A: represents the results of the Fc-tagged CD40-skipping 6 variant binding to mouse fibroblasts, stably transfected with full length human CD154. FIG. 18B: represents the results of the Fc-tagged CD40-WT binding to mouse fibroblasts, stably transfected with full length human CD154. FIG. 18C: represents the negative control, as the mouse fibroblast used do not express CD154 ligand. FIG. 18D summarizes the mean fluorescence shift, as plotted versus various concentrations of CD40 protein. FIG. 18E: represents the results of non-Fc-sCD40 Wt and CD40-skipping 6 binding to mouse fibroblasts, stably transfected with full length human CD154. FIG. 18F: represents the negative control, as the mouse fibroblast used do not express CD154 ligand. FIG. 18G: summarizes the mean fluorescence shift versus CD40 concentrations.

FIGS. 19A-E: demonstrate the Effect of the CD40 variant on RANATES secretion. The ability of the soluble skipping 6 protein, to compete with the CD40 membrane-bound receptor for binding to the secreted CD154 ligand, was tested in these experiments as compared to soluble wild-type CD40 protein. Detailed description of the experiments is provided in Example 5, below. FIG. 19A: represents the results of the control situation, where the mouse fibroblasts used in conjunction with the HPMC cells, were untransfected, thus did not express CD154 ligand. In FIG. 19B the mouse fibroblasts used in conjunction with the HPMC cells, were transfected to express CD154 ligand. INF and an appropriate commercially available anti-CD40 antibody were used as positive controls. The concentration of the administered CD40 proteins is indicated. FIG. 19C represents the control experiment for dose response assay, with mouse fibroblasts do not contain CD154 ligand. FIG. 19D represents the dose dependent inhibition of RANTES secretion by soluble CD40 proteins with mouse fibroblasts stably expressing the CD154 ligand. FIG. 19E demonstrates the inhibitory effect of the CD40-skipping 6 variant on RANTES secretion as verified by different independent purification batches.

FIG. 20 demonstrates the effect of the combinations of CD40 variants and their specific antibodies on RANTES Secretion.

FIGS. 21A-B demonstrate the CD40 WT-Fc and CD40-Skip6-Fc binding to CD154 using BIACORE. Detailed description of the experiments is provided in Example 6, below. FIGS. 21A and B shows the graphs indicating the association and dissociation profiles for various concentrations of the CD40 proteins, over the time-course of the experiment. Time is plotted on the X axis, with each concentration of CD40 protein represented by a separate plotted line raised one above the other. The CD40 wild type results are shown in FIG. 21A. The CD40-skipping 6 protein results are shown in FIG. 21B.

FIG. 22 demonstrates the RT-PCR results showing the mRNA expression of the CD40 variants in K652 cells.

FIG. 23 demonstrates the RT-PCR results showing the alteration of the expression pattern of the different variants of CD40 as a response to apoptosis in K562 cells.

FIG. 24 presents the percentage of the expression of each splice form out of the total CD40 expression levels for K562 cells treated with 20 micromolar Etoposide for various time intervals.

FIG. 25 demonstrates immunoblotting results of K562 cells treated with 25 μM etoposide for 8 hours to induce apoptosis, in order to measure the activation of caspases. The cells were lysed and immunobloted, and the PARP substrate for caspase-3, was probed using anti cleaved PARP antibody.

### DETAILED DESCRIPTION OF THE INVENTION

Active Ingredient of the pharmaceutical Composition

The active ingredient agent may be an agent comprising the full sequences of SEQ ID NO:1, fragment of at least 10 amino acids of SEQ ID NO:1 which contains at least four consecutive amino acids of the unique tail, sequences in which one or more of the amino acid residues in SEQ ID NO:1 is substituted with a conserved or non-conserved amino acid residue (preferably a conserved amino acid residue); or (ii) sequences in which one or more of the amino acid residues includes a constituent group (chemically modified), or (iii) sequences in which the “skipping 6” CD40 protein or peptide is fused with another compound, such as the F_{c} fragment of an antibody or a compound that increases the half-life of the protein (for example, polyethylene glycol (PEG)), or a moiety which serves as targeting means to direct the protein to its target tissue (such as an antibody or a fragment), or (iv) sequences in which additional amino acids are fused to the “skipping 6” CD40 protein or peptide. Such fragments, variants and derivatives are deemed to be within the scope of the invention for those skilled in the art from the teachings herein.

Substantially purified skipping 6 protein or peptide can be isolated from natural sources, produced by recombinant DNA methods or synthesized by standard protein synthesis techniques. Substantially purified functionally active fragments of skipping 6 protein, that comprise at least 10 amino acid residues including 4 amino acid residues of the unique tail sequence can be produced by processing proteins isolated from natural sources, or by recombinant DNA methods or synthesized by standard protein synthesis techniques.

Skipping 6 proteins or peptides which retains the ligand-binding domains of the original CD40 are capable of binding to its ligands (for example CD154) and decreasing in the individual the amounts of such free ligands available for binding to the original CD40. Thus, the proteins or peptides of the invention may act as “scavengers” of CD154, since they can bind those ligands and thus effectively lower the amount of said ligands in the circulation. Since skipping 6 is secreted it can exert its scavenging effect even in bodily fluids. This will effectively reduce the signaling of the CD40R-CD154 pathway.

Skipping 6 proteins or peptides of the invention are preferably soluble and bind to CD154 (also termed CD40 ligand). Thus, they compete with CD40 on cells associated with the immune system. Their presence reduces the signaling activity that occurs when CD40+ cells interact with CD154+ cells. The soluble alternatively spliced CD40 of the invention (skipping 6 proteins or peptides) thus modulates immune activity.

Accordingly, skipping 6 proteins or peptides be used as an active ingredient in a pharmaceutical composition used to modulate immune activity, particularly the immune activity associated with CD40-CD154 interactions.

In one embodiment, up to 20% of the amino acid of the native sequence have been replaced with a naturally or non-naturally occurring amino acid or with a peptidomimetic organic moiety; and/or up to 20% of the amino acids have their side chains chemically modified and/or up to 20% of the amino acids have been deleted, provided that at least 80% of the amino acids in the parent sequence is maintained unaltered, and provided that the amino acid maintains the biological activity of the parent sequence, in particular the CD40-L binding properties.

The active ingredient of the present invention preferably comprises the sequences of (i)-(iv) above. The active ingredient may have also an additional moiety/moieties attached to the C- and/or N-terminal, added for various purposes not related to the disruption CD40-CD154 interaction.

The composition may also comprise non-amino acid moieties, such as for example, hydrophobic moieties (various linear, branched, cyclic, polycyclic or hetrocyclic hydrocarbons and hydrocarbon derivatives) attached to the peptides of skipping 6, to improve penetration through membranes (for delivery purposes). In addition, various protecting groups, may be included, which are attached to the compound's terminals to decrease degradation; especially where the peptide is linear. Chemical (non-amino acid) groups may be included in order to improve various physiological properties such as penetration through membranes (moieties which enhance penetration through membranes or barriers); decreased degradation or clearance; decreased repulsion by various cellular pumps, improved immunogenic activities, improved various modes of administration (such as attachment of various sequences which allow penetration through various barriers such as BBB, through the gut, etc.); increased specificity, increased affinity, decreased toxicity, for imaging purposes and the like. The chemical groups may serve as various spacers, placed for example, between one or more of the above binding domains, so as to spatially position them in suitable orientation in respect of each other and in respect of the ligand.

The active ingredient of the invention may be linear or cyclic, and cyclization may take place by any means known in the art. Where the composition is composed predominantly of amino acids/amino acid sequences, cyclization may be N- to C-terminal, N-terminal to side chain and N-terminal to backbone, C-terminal to side chain, C-terminal to backbone, side chain to backbone and side chain to side chain, as well as backbone to backbone cyclization. Cyclization of the compound may also take place through the non-amino acid organic moieties. The association between the amino acid sequence component of the composition and other components of the composition may be by covalent linking, or by non-covalent complexion, for example, by complexion to a hydrophobic polymer, which can be degraded or cleaved producing a composition capable of sustained release; by entrapping the amino acid part of the composition in liposomes or micelles to produce the final composition of the invention. The association may be by the entrapment of the amino acid sequence within the other component (liposome, micelle) or the impregnation of the amino acid sequence within a polymer to produce the final composition of the invention.

Replacements/Substitutions

The term “wherein up to 20% of amino acids of the native sequence have been replaced” refers to substitution (conservative or non conservative) with a naturally or non-naturally occurring amino acid, or with a peptidomimetic organic moiety, The term concerns an amino acid sequence, which shares at least 80% of its amino acid with the native sequence as described in (i), (ii) or (iv) above, but some of the amino acids were replaced either by other naturally occurring amino acids, (both conservative and non-conservative substitutions), by non-naturally occurring amino acids (both conservative and non-conservative substitutions), or with organic moieties which serve either as true peptidomimetics (i.e. having the same steric and electrochemical properties as the replaced amino acid), or merely serving as spacers in lieu of an amino acid, so as to keep the spatial relations between the amino acid spanning this replaced amino acid. Guidelines for the determination of the replacements and substitutions are given in detail below. Preferably no more than 15%, 10% or 5% of the amino acids are replaced.

Chemical Modification

The term “chemically modified” refers to both the chemical modification of the side chains of the amino acids as well as to chemical modifications of the peptidic backbone. It also refers to a skipping 6 peptide which has the same type of amino acid residue, but a functional group has been added to its side chain. For example, the side chain may be phosphorylated, glycosylated, fatty acylated, acylated, iondiated or carboxyacylated. Other examples of chemical substitutions are known in the art and given below.

The replacement may be of at least one peptidic backbone has been altered to a non-naturally occurring peptidic backbone. For example, the bond between the N− of one amino acid residue to the C− of the next has been altered to non-naturally occurring bonds by reduction (to —CH2-NH—), alkylation (methylation) on the nitrogen atom, or the bonds have been replaced by amidic bond, urea bonds, or sulfonamide bond, etheric bond (—CH2-O—), thioetheric bond (—CH2-S—), or to —CS—NH—; The side chain of the residue may be shifted to the backbone nitrogen to obtain N-alkylated-Gly (a peptidoid).

Deletions

The term “deletions” refer to an amino acid sequence which maintains at least 20% of its parental amino acid content. Preferably no more than 10% of the amino acids are deleted and more preferably none of the amino acids are deleted.

The term “provided that at least 80% of the amino acids in the parent protein are maintained unaltered in the variants” the up to 20% substitution, up to 20% chemical modification and up to 20% deletions are combinatorial, i.e. the same variant may have substitutions, chemical modifications and deletions so long as at least 80% of the native amino acids are identical to those of the native sequence both as regards the nature of the amino acid residue and its position in the sequence In addition, the properties of the parent sequence, in binding to CD40 ligand (CD145), have to be maintained in the composition, typically at the same or higher level

Typically “essential amino acids” (essential for binding to the ligand) are maintained or replaced by conservative substitutions while non-essential amino acids may be maintained, deleted or replaced by conservative or non-conservative replacements. Essential amino acids are those indicated under E74, Y82, N86, D84, E114, E117 of the CD40 skipping exon 6 variant.

Addition of Groups

Fusion proteins of receptor molecules and the Fc of immunoglobulins have been shown to greater influence transmembrane signaling-related pathways than unfused receptor molecules, presumably by creating receptor dimers which are more stable than monomers (K M Mohler, et al., J. Immunol, 151, (3) 1548-1561, 1993). Addition of an Fc chain to various CD40 proteins has been shown to increase the lifetime (T1/₂) of the construct, and to simplify the protein extraction procedure.

Where the composition of the invention is a linear molecule, it is possible to place in any of its terminals various functional groups. The purpose of such a functional group may be for the improvement of the CD40 ligand binding of the composition. The functional groups may also serve for the purpose of improving the activity of the composition in a manner such as: improvement in stability, penetration (through cellular membranes or barriers), tissue localization, efficacy, decreased clearance, decreased toxicity, improved selectivity, improved resistance to repletion by cellular pumps, and the like. For convenience sake the free N-terminal of one of the sequences contained in the compositions of the invention will be termed as the N-terminal of the composition, and the free C-terminal of the sequence will be considered as the C-terminal of the composition (these terms being used for convenience sake). Either the C-terminus or the N-terminus of the sequences, or both, can be linked to a carboxylic acid functional groups or an amine functional group, respectively.

Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moietyfor transport across cellular membranes”.

These moieties can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 12:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH 3)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Replacements by Peptidomimetic Compositions

The replacement may be also by a peptidomimetic organic moiety.

A “peptidomimetic organic moiety” can be substituted for amino acid residues in the composition of this invention both as conservative and as non-conservative substitutions. These peptidomimetic organic moieties can replace amino acid residues, amino acids or act as spacer groups within the peptides in lieu of deleted amino acids. The peptidomimetic organic moieties often have steric, electronic or configurational properties similar to the replaced amino acid and such peptidomimetics are used to replace amino acids in the essential positions, and are considered conservative substitutions. However such similarities are not necessarily required. The only restriction on the use of peptidomimetics is that the composition retains its physiological activity as compared to sequence regions identical to those appearing in the native protein.

Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 22: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 22:5081-5082 (1988) as well as Kemp et al., Tetrahedron Lett. 29:5057-5060 (1988), Kemp et al., Tetrahedron Lett. 22:4935-4938 (1988) and Kemp et al., J. Org. Chem. 54:109-115 (1987). Other suitable peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

Chemical Modification

In the present invention the side amino acid residues appearing in the native sequence may be chemically modified, i.e. changed by addition of functional groups. The modification may be in the process of synthesis of the molecule, i.e. during elongation of the amino acid chain and amino acid, i.e. a chemically modified amino acid is added. However, chemical modification of an amino acid when it is present in the molecule or sequence (“in situ” modification) is also possible.

The amino acid of any of the sequence regions of the molecule can be modified (in the peptide conceptionally viewed as “chemically modified”) by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

Cyclization of the Molecule

The present invention also includes cyclic compounds that are cyclic molecules.

A “cyclic molecule” refers, in one instance, to a compound of the invention in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the sequence present therein, preferably the side chain of the C-terminal amino acid. For example, an amide can be formed between the nitrogen atom at the N-terminus and the carbonyl carbon in the side chain of an aspartic acid or a glutamic acid. Alternatively, the compound can be cyclized by forming a covalent bond between the carbonyl at the C-terminus of the compound and the side chain of a suitable amino acid in the sequence contained therein, preferably the side chain of the N-terminal amino acid. For example, an amide can be formed between the carbonyl carbon at the C-terminus and the amino nitrogen atom in the side chain of a lysine or an ornithine. Additionally, the compound can be cyclized by forming an ester between the carbonyl carbon at the C-terminus and the hydroxyloxygen atom in the side chain of a serine or a threonine.

“Cyclized” also refers to forming a ring by a covalent bond between the side chains of two suitable amino acids in the sequence present in the compound, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a compound can be cyclized with a linking group between the two termini, between one terminus and the side chain of an amino acid in the compound, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

The composition of the present invention can be administered parenterally. Parenteral administration can include, for example, systemic administration, such as by intramuscular, intravenous, subcutaneous, or intraperitoneal injection. Compositions that resist proteolysis can be administered orally, for example, in capsules, suspensions or tablets. The composition can also be administered by inhalation or insufflations or via a nasal spray.

The active ingredient of the invention can be administered to the individual in conjunction with an acceptable pharmaceutical carrier as part of a pharmaceutical composition for treating the diseases discussed above. Suitable pharmaceutical carriers may contain inert ingredients which do not interact with the active ingredients. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Methods for encapsulating compositions (such as in a coating of hard gelatin or cyclodextran) are known in the art (Baker et. al., Controlled Release of Biological Active Agents, John Wiley and Sons, 1986). The formation may be also resources for administration to bone, or in the form of salve, solution, ointment, etc. for topical administration.

The pharmaceutical compositions may also be administered in conjunction with other modes of therapy routinely used in the treatment of the diseases specified.

A “therapeutically effective amount” is the quantity of active ingredient which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment.

Preparation of the Active Ingredients

One having ordinary skill in the art can isolate the nucleic acid molecule that encodes the skipping 6 CD40 protein, and insert it into an expression vector using standard techniques and readily available starting materials. Use can be made of a recombinant expression vector that comprises a nucleotide sequence encoding for the amino acid sequence of SEQ ID NO: 1, or the sequence (ii) (iii), (iv) as defined above. These recombinant expression vectors are useful for transforming hosts to prepare recombinant expression systems for preparing the pharmaceutical composition of the invention.

Preparation by recombinant Methods

As will be understood by those of skill in the art, it may be advantageous to use nucleotide sequences possessing codons other than those which naturally occur in the human genome. Codons preferred by a particular prokaryotic or eukaryotic host (Murray, E. et al. Nuc Acids Res., 17:477-508, (1989)) can be selected, for example, to increase the rate of variant product expression or to produce recombinant RNA transcripts having desirable properties, such as a longer half-life, than transcripts produced from naturally occurring sequence.

The nucleic acid sequences used to produce the amino acid sequence of the present invention can be engineered in order to alter the skipping 6 CD40 protein/peptide sequences for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, etc.

For producing the protein or peptide used by the present invention recombinant constructs comprising the sequence as broadly described above. The constructs may comprise a vector, such as a plasmid or viral vector, into which nucleic acid sequences coding for the protein/peptide of the invention have been inserted, in a forward or reverse orientation. In a preferred aspect of this embodiment, the constructs further comprise regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al., Molecular Cloning a Laboratory Manual, Second Ed. Cold Spring Harbor Press (1989) which is incorporated herein by reference.

The preparation may be achieved by host cells which are genetically engineered with the above vectors and the production of the product skipping 6 protein/peptide of the invention by recombinant techniques. Host cells are genetically engineered (i.e., transduced, transformed or transfected) with the above vectors which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the expression of the variant nucleic acid sequence. The culture conditions, such as temperature, pH and the like, are those previously used with the host cell selected for expression and will be apparent to those skilled in the art.

The host cells used in the preparation may comprise the recombinant expression vector that includes a nucleotide sequence that encodes a skipping 6 CD40 protein of SEQ ID NO:1, and fragments and variants thereof. Host cells for use in well known recombinant expression systems for production of proteins are well known and readily available. Examples of host cells include bacteria cells such as E. coli, yeast cells such as S. cerevisiae, insect cells such as S. frugiperda, non-human mammalian tissue culture cells, chinese hamster ovary (CHO) cells and human tissue culture cells such as HeLa cells.

The nucleic acid sequences used to prepare the peptide/proteins may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E. coli, lac or trp promoter, the phage lambda PL promoter, and other promoters known to control expression of genes in prokaryotic or eukaryotic cells or their viruses. The expression vectors also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vectors containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sj9; animal cells such as CHO, COS, HEK 293 or Bowes melanoma; adenoviruses; plant cells, etc. The selection of an appropriate host is deemed to be within the scope of those skilled in the art from the teachings herein. The invention is not limited to any particular host cells which can be employed.

One having ordinary skill in the art can use commercial expression vectors and systems or others to produce the CD40 product of the invention using routine techniques and readily available starting materials. Thus, the desired proteins can be prepared in both prokaryotic and eukaryotic systems, resulting in a spectrum of processed forms of the protein. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989).

A wide variety of eukaryotic hosts are also now available for production of recombinant foreign proteins. As in bacteria, eukaryotic hosts may be transformed with expression systems which produce the desired protein directly, but more commonly signal sequences are provided to effect the secretion of the protein. Eukaryotic systems have the additional advantage that they are able to process introns which may occur in the genomic sequences encoding proteins of higher organisms. Eukaryotic systems also provide a variety of processing mechanisms which result in, for example, glycosylation, carboxy-terminal amidation, oxidation or derivatization of certain amino acid residues, conformational control, and so forth.

Commonly used eukaryotic systems include, but are not limited to, yeast, fungal cells, insect cells, mammalian cells, avian cells, and cells of higher plants. Suitable promoters are available which are compatible and operable for use in each of these host types as well as are termination sequences and enhancers, e.g. the baculovirus polyhedron promoter. As above, promoters can be either constitutive or inducible. For example, in mammalian systems, the mouse metallothionein promoter can be induced by the addition of heavy metal ions.

The particulars for the construction of expression systems suitable for desired hosts are known to those in the art. Briefly, for recombinant production of the protein, the DNA encoding the polypeptide is suitably ligated into the expression vector of choice. The DNA is operably linked to all regulatory elements which are necessary for expression of the DNA in the selected host. One having ordinary skill in the art can, using well known techniques, prepare expression vectors for recombinant production of the polypeptide.

The expression vector including the DNA that encodes the CD40 skipping 6 protein, fragment or homolog, preferably including DNA coding for the Fc fragment attached to the CD40 skipping 6 protein, is used to transform the compatible host which is then cultured and maintained under conditions wherein expression of the foreign DNA takes place. The protein of the present invention thus produced is recovered from the culture, either by lysing the cells or from the culture medium as appropriate and known to those in the art. One having ordinary skill in the art can, using well known techniques, isolate the CD40 product that is produced using such expression systems. The methods of purifying the CD40 skipping 6 protein from natural sources using antibodies which specifically bind to the skipping 6 protein, may be equally applied for purifying the product produced by recombinant DNA methodology.

Examples of genetic constructs include the skipping 6 CD40 protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes the skipping 6 protein from readily available starting materials.

In bacterial systems, a number of expression vectors may be selected depending upon the use intended for the CD40 product. For example, when large quantities of CD40 skipping 6 splice variant product are needed, such as for the induction of antibodies, vectors which direct high level expression of fusion proteins that are readily purified may be desirable. Such vectors include, but are not limited to, multifunctional E. coli cloning and expression vectors such as Bluescript(R) (Stratagene), in which the CD40 splice variant polypeptide coding sequences may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); and the like. In some embodiments, for example, one having ordinary skill in the art can, using well known techniques, insert such DNA molecules into a commercially available expression vector for use in well known expression systems. For example, the commercially available plasmid pSE420 (Invitrogen, San Diego, Calif.) may be used for production of collagen in E. coli.

In the yeast Saccharomyces cerevisiae a number of vectors containing constitutive or inducible promoters such as alpha factor, alcohol oxidase and PGH may be used. For reviews, see Ausubel et al. (Supra) and Grant et al., (Methods in Enzymology 153:516-544, (1987)). The commercially available plasmid pYES2 (Invitrogen, San Diego, Calif.) may, for example, be used for production in S. cerevisiae strains of yeast.

In cases where plant expression vectors are used, the expression of a sequence encoding variant products may be driven by any of a number of promoters. For example, viral promoters such as the ³⁵S and 19S promoters of CaMV (Brisson et al., Nature 310:511-514. (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

CD40 splice variants products may also be expressed in an insect system. In one such system, Autographa californica nuclear polyhedrosis virus (AcNPV) is used as a vector to express foreign genes in Spodoptera frugiperda cells or in Trichoplusia larvae. The CD40 skipping 6 coding sequence may be cloned into a nonessential region of the virus, such as the polyhedrin gene, and placed under control of the polyhedrin promoter. Successful insertion of the skipping 6 coding sequence will render the polyhedrin gene inactive and produce recombinant virus lacking coat protein coat. The recombinant viruses are then used to infect S. frugiperda cells or Trichoplusia larvae in which variant protein is expressed (Smith et al., J. Virol. 46:584, (1983); Engelhard, E. K. et al., Proc. Nat. Acad. Sci. 21:3224-7, (1994)). The commercially available MAXBACJ complete baculovirus expression system (Invitrogen, San Diego, Calif.) may, for example, be used for production in insect cells.

In mammalian host cells, a number of viral-based expression systems may be utilized. In cases where an adenovirus is used as an expression vector, the skipping 6 CD40 coding sequences may be ligated into an adenovirus transcription/translation complex consisting of the late promoter and tripartite leader sequence. Insertion in a nonessential E1 or E3 region of the viral genome will result in a viable virus capable of expressing variant protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells. The commercially available plasmid pcDNA I (Invitrogen, San Diego, Calif.) may, for example, be used for production in mammalian cells such as Chinese Hamster Ovary cells.

Specific initiation signals may also be required for efficient translation of product coding sequences. These signals include the ATG initiation codon and adjacent sequences. In cases where the CD40 sequence, its initiation codon and upstream sequences are all inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Differ., 20:125-62, (1994); Bittner et al., Methods in Enzymol 153:516-544, (1987)).

The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “A pre-pro” form of the protein may also be important for correct insertion, folding and/or function. Different host cells such as CHO, HeLa, MDCK, 293, WI38, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express skipping 6, may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant colonies of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt-cells, respectively. Also, antimetabolite, antibiotic or herbicide resistance can be used as the basis for selection; for example, dhfr which confers resistance to methotrexate (Wigler M., et al., Proc. Natl. Acad. Sci. 77:3567-70, (1980)); npt, which confers resistance to the aminoglycosides neomycin and G-418 (Colbere-Garapin, F. et al., J. Mol. Biol., 150: 1-14, (1981)) and als or pat, which confer resistance to chlorsulfuron and phosphinotricin acetyltransferase, respectively (Murry, Supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et al., Methods Mol. Biol., 55:121-131, (1995)).

Host cells transformed with nucleotide sequences encoding the skipping 6 or its fragments, may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors can be designed with signal sequences which direct secretion of the skipping 6 CD40 product through a prokaryotic or eukaryotic cell membrane.

The present invention encompasses use of a transgenic non-human mammal that comprises the recombinant expression vector that comprises a nucleic acid sequence encoding for the CD40 splice variant of amino acid sequence SEQ ID NO:1; and fragments and homologues thereof. Transgenic non-human mammals useful to produce recombinant proteins are well known as are the expression vectors necessary and the techniques for generating transgenic animals. Typically, the transgenic animal comprises a recombinant expression vector in which the nucleotide sequence that encodes the skipping 6 CD40 protein of the invention is operably linked to a mammary cell specific promoter whereby the coding sequence is only expressed in mammary cells and the recombinant protein expressed is recovered from the animal's milk. One having ordinary skill in the art using standard techniques, such as those taught in U.S. Pat. No. 4,873,191 issued Oct. 10, 1989 to Wagner and U.S. Pat. No. 4,736,866 issued Apr. 12, 1988 to Leder, both of which are incorporated herein by reference, can produce transgenic animals which produce the CD40 product of the present invention. Preferred animals are rodents, particularly, rats and mice, or goats.

In some embodiments, the skipping 6 protein may be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.).

The inclusion of a protease-cleavable polypeptide linker sequence between the purification domain and the skipping 6 protein, is useful to facilitate purification. One such expression vector provides for expression of a fusion protein compromising a variant polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, then disrupted by physical or chemical means, and the resulting crude extract retained for further purification. Microbial cells employed in expression of proteins can by disrupted by any convenient method, including freeze-thaw cycling, sonication, mechanical disruption, or use of cell lysing agents, or other methods, which are well know to those skilled in the art.

Purification of Recombinant Produced Peptide/Proteins

The CD40 skipping 6 product can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps. In some embodiments, antibodies may be used to isolate the skipping 6 proteins.

Production hy Synthesizers

In addition to producing these proteins by recombinant techniques, automated peptide synthesizers may also be employed to produce the CD40 skipping 6 proteins, fragments or homologues of the invention. Such techniques are well known to those having ordinary skill in the art and are useful if derivatives which have substitutions not provided for in DNA-encoded protein production. CD40 skipping 6, fragments and portions of the products may be produced by direct peptide synthesis using solid-phase techniques (cf. Stewart et al., (1969) Solid-Phase Peptide Synthesis, WH Freeman Co, San Francisco; Merrifield J., J. Am Chem. Soc., 85:2149-2154, (1963)). In vitro peptide synthesis may be performed using manual techniques or automation. Automated synthesis may be achieved, for example, using Applied Biosystems 431A Peptide Synthesizer (Perkin Elmer, Foster City, Calif.) in accordance with the instructions provided by the manufacturer. Fragments of the skipping 6 CD40 protein may be chemically synthesized separately and combined using chemical methods to produce the full length molecule.

Immune Rejection of Transplanted Organs; Treatment with CD40 Splice Variants

Organ transplant is a treatment option that is often the only possibility to save human life under conditions in which the individual's own organ has become severely compromised. Organs transplanted include kidneys, hearts, bone marrow and livers (often only a portion of the liver is transplanted). Because the tissue of the transplanted organ comes from a donor who may have different immune-reactive antigens (which is generally true for all donors except for identical twins), organ rejection frequently occurs unless treated with immune-suppressive drugs. However, these drugs also may cause many unwanted side effects. Without these drugs, the recipient of the transplanted organ will almost certainly undergo a fatal immune reaction.

The exact mechanism by which organ rejection occurs is still being researched, but ultimately it involves the recognition of the transplanted organ as being “non-self” and hence a target for attack by the body's immune system. This response may be augmented by the damage that occurs during surgery, which results in an inflammation reaction. Another potentially important component is the fact that humans are typically exposed to many antigens in a lifetime, which may also augment transplant rejection through a process known as heterologous immunity (Adams et al, Immunology Review, 2003, vol 196, p. 147). As described herein, the interaction of CD40 with CD154 is an important component of the immune process, such that blocking this interaction is expected to prevent or at least ameliorate rejection of the transplanted organ.

CD40 variants according to the present invention may be administered in any manner which is medically acceptable. Depending on the specific circumstances, local or systemic administration may be desirable. Preferably, the compound is administered via a parenteral route such as by an intravenous, intraarterial, subcutaneous, intramuscular, intraorbital, intraventricular, intraperitoneal, subcapsular, intracranial, topical, intraspinal, intradermal, subdermal or intranasal injection, infusion or inhalation route. The compound may also be administered via an oral or an enteral route. The compound also may be administered by implantation of an infusion pump, or a biocompatible or bioerodable sustained release implant, into the recipient host, either before or after implantation of donor tissue. Alternatively, certain compounds of the invention, or formulations thereof, may be appropriate for oral or enteral administration. Still other compounds of the invention will be suitable for topical administration to asuitable tissue surface, such as a surgical site, a wound site (e.g. an abrasion or a bum) or any other tissue surface which permits uptake of the compound by the body of the recipient.

For skin grafts, topical administration (including administration to graft beds and wound sites), subdermal application, including local injection, intradermal and subcutaneous application, and other methods that allow absorption of the compound into the graft bed are preferred routes of administering the anti-CD40L compound to a skin graft recipient; systemic administration is also possible.

The amount of and frequency of dosing for a CD40 variant according to the present invention to be administered to a patient for a given immune complex disease is within the skills and clinical judgement of ordinary practitioners of the tissue transplant arts, such as transplant surgeons. The general dosage and administration regime is established by preclinical and clinical trials, which involve extensive but routine studies to determine the optimal administration parameters of the compound. These dosages may optionally be varied for different recipient hosts based on a variety of considerations, such as the individual's age, medical status, weight, sex, and concurrent treatment with other pharmaceuticals. Determining the optimal dosage and administration regime for each CD40 variant according to the present invention that is used to inhibit graft rejection is a routine matter for those of skill in the pharmaceutical and medical arts.

Generally, the frequency of dosing may be determined by an attending physician or similarly skilled practitioner, and might include periods of greater dosing frequency, such as at daily or weekly intervals, alternating with periods of less frequent dosing, such as at monthly or longer intervals.

According to preferred embodiments of the present invention, the CD40 splice variant according to the present invention is preferably administered serially or in combination with conventional anti-rejection therapeutic agents or drugs such as, for example, corticosteroids or immunosuppressants. Combination therapies according to this invention for treatment of graft and/or transplant rejection include but are not limited to combining a CD40 splice variant according to the present invention with agents targeted to B cells, such as anti-CD19, anti-CD28 or anti-CD20 antibody (unconjugated or radiolabeled), IL-14 antagonists, LJP394 (LaJolla Pharmaceuticals receptor blocker), IR-1116 (Takeda small molecule) and anti-Ig idiotype monoclonal antibodies. Alternatively, the combinations may include T cell/B cell targeted agents, such as CTLA4-Ig, cytokine antagonists such as IL-2 antagonists, IL-4 antagonists, IL-6 antagonists, and IL-15 antagonists, receptor antagonists, anti-CD80/CD86 and anti-B7 monoclonal antibodies, TNF antagonists, LFA1/ICAM antagonists, VLA4/VCAM antagonists, LT/LT.beta., CD2/LFA3 antagonists, brequinar and IL-2 toxin conjugates (e.g., DAB), prednisone, anti-CD3 mAb such as OKT3, mycophenolate mofetil (MMF), cyclophosphamide, CD45RB antagonists, rapamycin, and other immunosuppressants such as calcineurin signal blockers, including without limitation, tacrolimus (FK506). Combinations may also include T cell targeted agents, such as CD4 antagonists, CD2 antagonists and IL-12.

For maintenance of graft/transplant integration, or in a period following suppression of an acute episode of graft/transplant rejection, a maintenance dose of CD40 splice variant, alone or in combination with a conventional anti-rejection agent is administered, if necessary. Subsequently, the dosage or the frequency of administration, or both, may be reduced. Where no sign of graft/transplant rejection is evident, treatment might cease, with vigilant monitoring for signs of graft/transplant rejection. In other instances, as determined by the ordinarily skilled practitioner, occasional treatment might be administered, for example at intervals of four weeks or more. Recipient hosts may, however, require intermittent treatment on a long-term basis upon any recurrence of disease symptoms.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Production of Skip 6 Polyclonal Antibody

Rabbits were Immunized with KLH Conjugated 95% Purified 15aa Peptide (ESPGGDPHHLRDPVC, SEQ ID NO: 3) taken from the Unique Tail of Skip 6 Variant (SEQ ID NO: 24).

The anti-CD40-skipping 6 antibodies were then purified from rabbit serum by ammonium sulfate precipitation. Briefly, a saturated solution of ammonium sulfate was prepared by adding 380 gr to 500 ml water and boiling the solution. The serum was thawed and centrifuged at 10,000 rpm, 4° C. for 5 min. One vol. PBS was added to each vol. serum, and stirred at 4° C.

One volume of saturated ammonium sulfate was then added under stirring for at least 2 hours on ice. The solution was centrifuged 15 min. at 10,000 rpm at 4° C. to precipitate IgG. The pellet was resuspended in 5 ml PBS and dialyzed overnight at 4° C. against PBS+0.05% azide. The precipitated serum was filtered with a 0.45 μm filter.

Affinity purification was then performed with the peptide against which the respective antibodies were raised as described above, in an immunoaffinity column, linked to sulfolink beads (Pierce # 20401). The column was prepared according to manufacturer's instructions. The serum to be purified was mixed with sulfolink beads and incubated under gentle shaking (1 h at room temperature and 2 h at 40 C), after which the beads were packed into a column.

The column was washed with TRIS 100 mM, followed by binding buffer containing 0.5M NaCl. The IgG was eluted by applying elution buffer: 0.1M Glycine pH3 (fraction size: 0.5 ml), followed by phosphate buffer 100 mM pH11 to elute another fraction of IgG. In order to neutralize acidic or basic pH, 1/10 volume TRIS 1M pH8 was added to collecting tubes before addition of elution buffer to the column. The antibodies were dialyzed overnight against a buffer of PBS and 0.025% azide, and then frozen for storage.

Measurement of Peptide Concentration:

2 mg of lyophilized peptide were diluted into 1 ml DDW and the absorbance at A280 was measured. 2 ml of slurry sulfolink gel at room temperature were added to a column and the buffer was drained.

The column was equilibrated with 6 volumes of coupling buffer, containing 50 mM Tris, 5 mM EDTA pH 8.5. 5 ml coupling buffer containing 1 mM β-mercaptoehtanol (3.5 μl β-mercaptoehtanol 98% to 50 ml TRIS buffer) were added to the peptide, following the addition of the beads and mix at RT for 15 min. Additional incubation for another 30 min at RT without mixing followed.

Next, the buffer was drained, kept and measured for absorbance to check for unbound peptide. The absorbance was measured as follows: A280=0.2.

Blocking was performed by the addition of β-mercaptoethanol (100 mM) to the coupling buffer (35 μl β-mercaptoethanol to 5 ml buffer) and mixing at RT for 15 min, following incubation at RT for 30 min without mixing. Following the washing with the coupling buffer without β-mercaptoehtanol and then with NaCl 1M, the column was kept in PBS or TRIS containing 0.05% NaN3.

Purification using FPLC:

Before the first use of the columns, all of the buffers used for purification were passed through the column, in order to remove any remaining impurities in the beads.

Into each collecting tube, 1/10 collecting volume of TRIS 1M pH 8 on ice were added. The column was washed and kept in the binding buffer (PBS) until the antibody-containing sample was loaded. The precipitated serum was filtered, mixed with sulfolink beads and incubated under gentle shaking (1 h at R.T. and 2 h at 4° C.). The beads were packed into the column and washed with TRIS 100 mM, and were then washed with binding buffer containing 0.5M NaCl. The eluted fractions were kept for further analyses (to check they do not contain the Ab). Purified_IgG was eluted by applying elution buffer: 0.1M Glycine pH3 (fractions size: 0.5 ml). Each collected fraction was mixed immediately with the 1M TRIS buffer (1:10) in the collecting tube. The column was washed with binding buffer. Phosphate buffer 100 mM pH11 was applied to elute another fraction of IgG. The absorption of the relevant fractions was measured at OD A₂₈₀ (GeneQuant: use the DNA channel) (calculation of concentration: A₂₈₀=n; C=n/1.35 mg/ml) and put to dialysis o.n against PBS+0.025% azide.

The column was regenerated and stored as follows: washed with PBS 50 mM+0.025% azide and stored at 4° C.

The antibodies were stored as follows: aliquoted and stored in one of three different solutions:
4° C. with 1% BSA and sodium azide (0.025%)
−20° C. with 1% BSA, 50% glycerol and sodium azide (0.025%)
−70° C. with 1% BSA and sodium azide (0.025%)

### Example 2

Cloning of CD 40 Skipping6 Variant and the Wild Type CD40

For of all the constructed transfer vectors, the backbone was the pTen21 plasmid whose full-length sequence is given in SEQ ID NO:4. The plasmid map and its multiple cloning site sequences are given in FIG. 1:

I—Unfused Constructs:

1—Construction of pTen21-CD40 wtEC Vector:

The CD40 wild type extracellular domain (CD40 wtEC) sequence was amplified by PCR using the following primers (the transmembrane domain of the wild type CD40 protein was excluded, therefore this CD40 wild type protein, upon translation, will be secreted).

(SEQ ID NO: 5)

5′-ACTAgATATCATggT TCgTCTgCCTCTgCAgT-3′
(Called 40wt5′)

(SEQ ID NO: 6)

5′-AAgCAgATCTTATCTCAgCCgATCCTgggg-3′
(Called 40wt3′)

The PCR reaction was carried out using the ISIS DNA polymerase (Qbiogene Cat# EPSIS100).

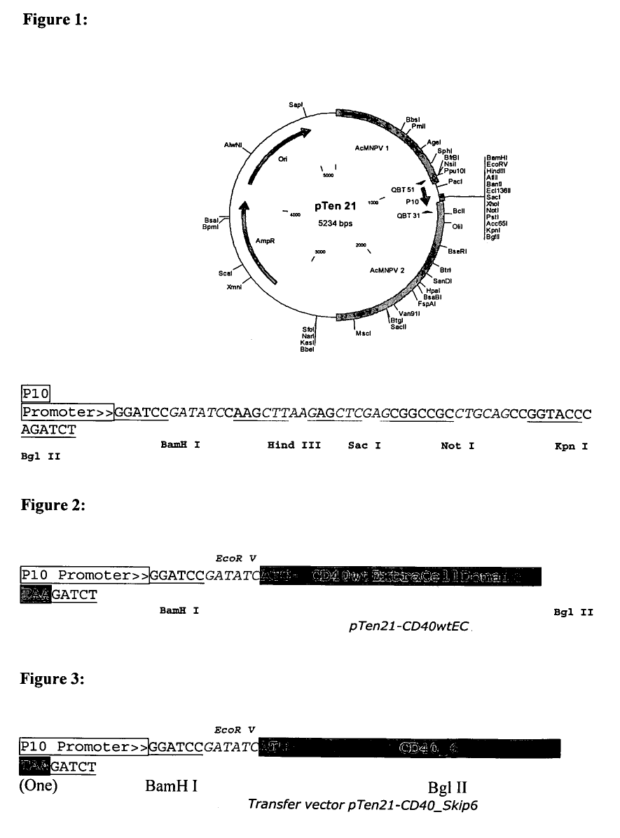

The amplified fragment of about 600 bp was digested with EcoRV and BglII and ligated to pTen21 vector previously cut with the same enzymes to obtain the vector as described in FIG. 2.

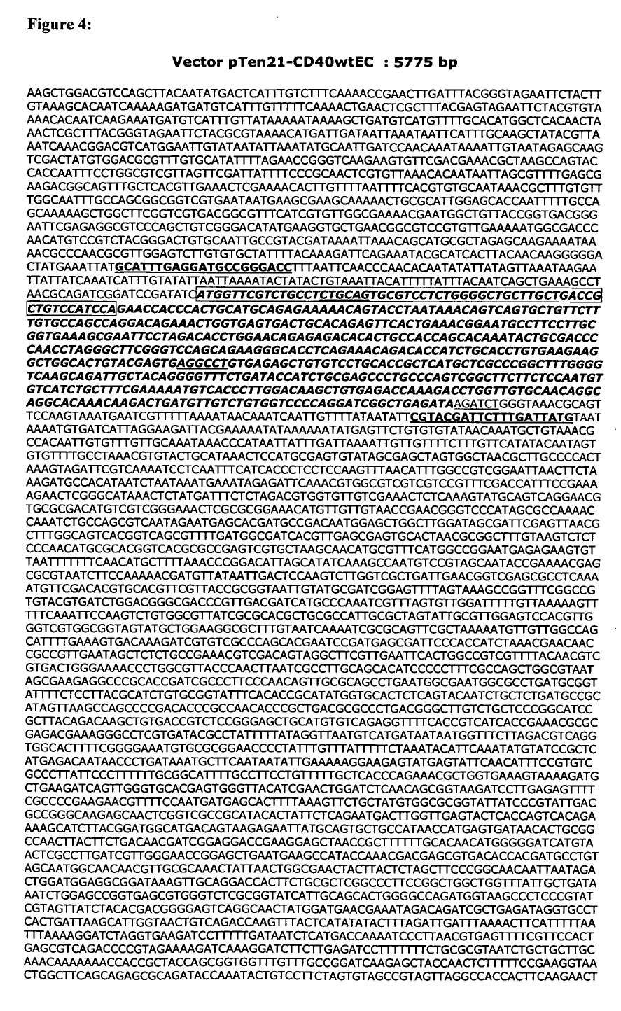

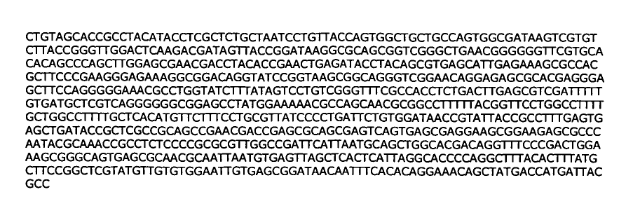

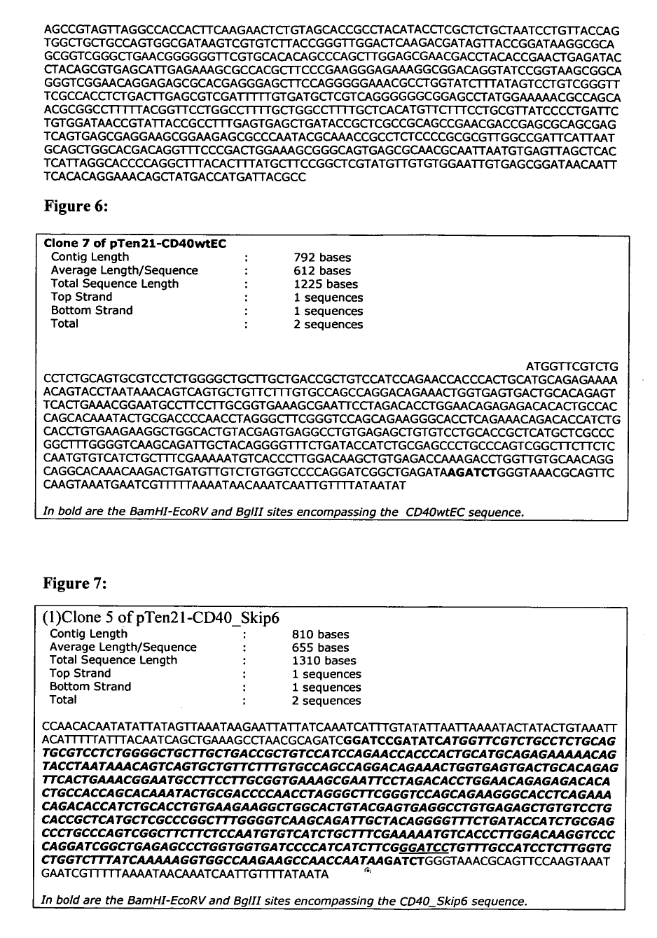

Among the sequenced recombinant constructs, clone number 7 was selected. The bi-directional sequencing was performed using the upstream primer OQBT51 (5′-GCATTTGAGGATGCCGGGACC-3′, SEQ ID NO: 7) and the downstream primer OQBT31 (5′-CATAATCAAAGAATCGTACG-3′, SEQ ID NO:8). The positions of these 2 primers are indicated (bold & underlined) on the vector sequences (SEQ ID NO: 12) in FIG. 4. In FIG. 4 the primers are in bold and underlined, in bold italics is the CD40 wtEC coding sequence. Also shown are the BamHI-EcoRV and BglII sites, and the signal peptide-encoding sequence is shown in the rectangle. The sequence determined for Clone 7 is the described in SEQ ID NO:9 and in FIG. 6, featuring the BamHI-EcoRV and BglII sites marked in bold and the CD40 wtEC sequence.

2-Construction of pTen21-CD40-Skip6 Vector:

Using the pET28a-CD40_{—}6 plasmid, a sequence extending from the internal StuI site to the Stop codon of CD40_Skip6 variant was amplified by PCR. For this purpose, the following primers were used:

(SEQ ID NO: 10)

5′-CACCATCTgCACCTgTgAAg-3′
(Stu40)

(SEQ ID NO: 11)

5′-AATgAgATCTTATTggTTggCTTCTTggCC-3′
(Called 4063′)

The amplified fragment of about 320 bp was digested with StuI and BglII and ligated to the previously constructed pTen21-CD40 wtEC clone 7 vector cut with the same enzymes to obtain the vector as described in FIG. 3.

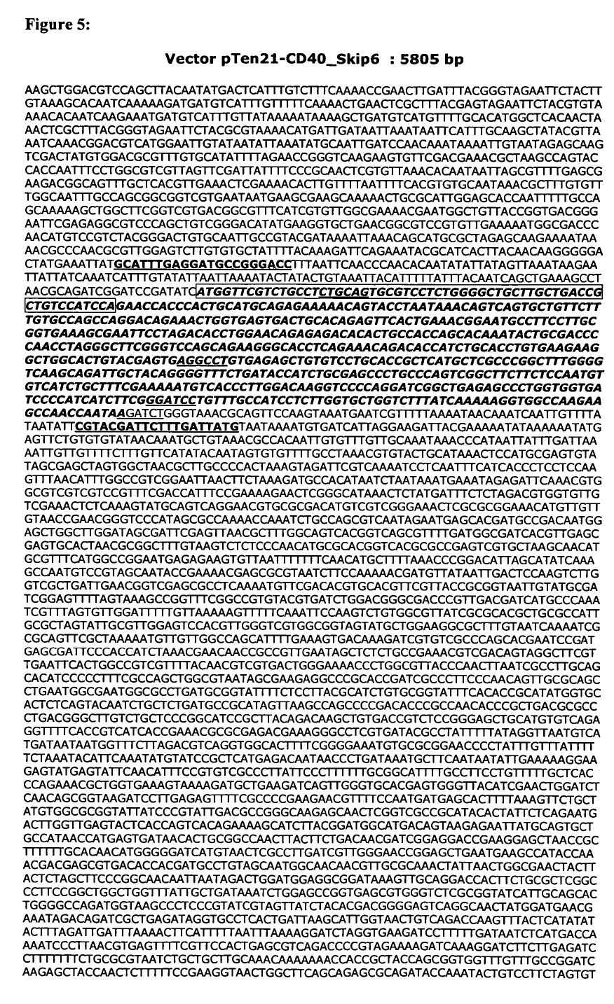

A series of recombinant plasmids were selected and sequenced using the OQBT51 (SEQ ID NO: 7) and OQBT31 (SEQ ID NO: 8) primers. Among those, clones 3, 5 and 29 presented 100% homology when aligned with the corresponding portion of the expected sequence of pTen21-CD40_Skip6 vector (SEQ ID NO:13) shown in FIG. 5. In FIG. 5 the primers are in bold and underlined. Also shown in bold italics is the CD40_Skip6 coding sequence, the BamHI-EcoRV and BglII sites, and the signal peptide-encoding sequence, which is presented in the rectangle. The sequence obtained for clone 5 is described in SEQ ID NO: 14 and in FIG. 7, featuring the BamHI-EcoRV and BglII sites marked in bold, encompassing the CD40_Skip6 sequence, marked in red.

3—Construction of pTen21-CD40 wtEC-F Vector

Fused constructs were created, in which the Fc chain of Immunoglobulin IgG1 was fused downstream from the CD40 protein (either downstream of Skipping 6, or downstream of the WT CD40). Fusion proteins of receptor molecules and the Fc of immunoglobulins are known to have greater influence on transmembrane signaling-related pathways than unfused receptor molecules, presumably by creating receptor dimers which are more stable than monomers (K M Mohler, et al., J. Immunol, 151, (3) 1548-1561, 1993). Addition of an Fc chain to various CD40 proteins has been shown to increase the lifetime (T1/₂) of the construct, and to simplify the protein extraction procedure.

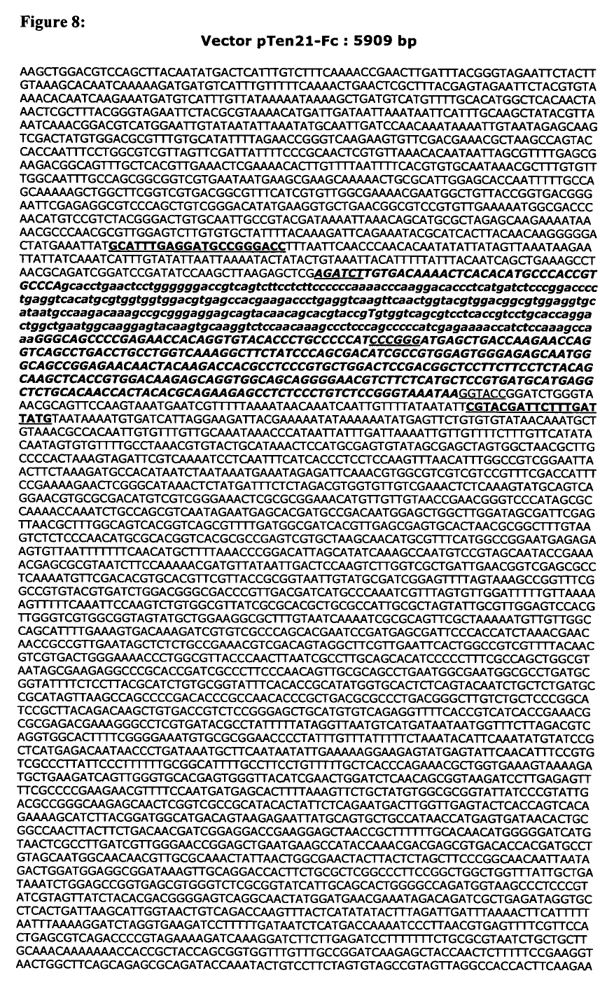

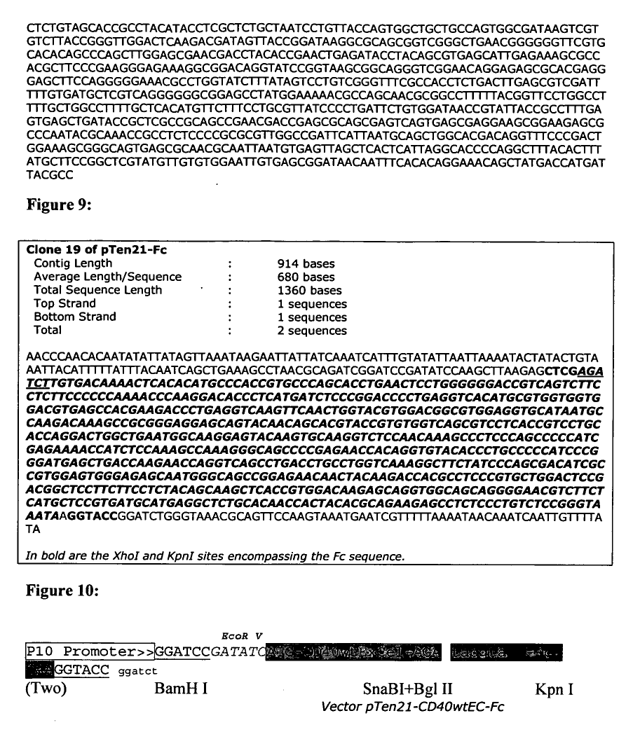

To create the Fc-fused CD40 vectors, the pTen21-Fc vector was used. The sequence of the full length pTen21-Fc vector is shown in SEQ ID NO: 15 and in FIG. 8, where the OQBT primers are in bold and underlined; the Fc sequence is shown in bold italics. The polylinker is also shown. The Fc sequence within the pTen21-Fc vector is shown in SEQ ID NO: 16 and in FIG. 9, where in bold are the XhoI and KpnI sites encompassing the Fc sequence, which is shown in bold italics.

To construct the pTen21-CD40 wtEC-Fc fusion transfer vector, the CD40 wild type extracellular domain sequence has been amplified by PCR using the following primers:

(SEQ ID NO: 5)

5′-ACTAgATATCATggT TCgTCTgCCTCTgCAgT-3′
(40wt5′)

(SEQ ID NO: 17)

5′-CACAAgATCTgggCTCTACgTATCTCAgCCgATCCTgggg-3′
(Called 40Fc3′)

The amplified fragment of about 610 bp has been digested with EcoRV and BglII and ligated to pTen21-Fc clone 19 vector also cut with the same enzymes to obtain the vector as described in FIG. 10.

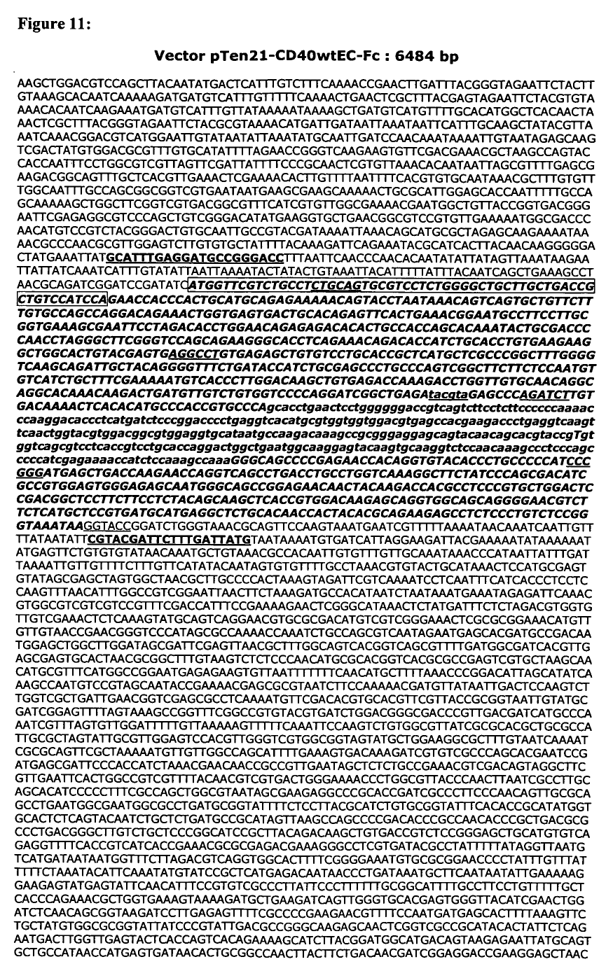

A series of recombinant plasmids have been selected and sequenced using the OQBT51 (SEQ ID NO:7), OQBT31 (SEQ ID NO:8) and the internal Stu40 (SEQ ID 10) primer. Among them, clones 30 and 37 presented 100% homology when aligned with the corresponding portion of the expected sequence of pTen21-CD40 wtEC-Fc vector shown in SEQ ID NO: 18 and in FIG. 11, where primers are in bold and underlined. The CD40 wtEC-Fc fusion sequences are shown in bold italics and are separated by the tacgta sequence.

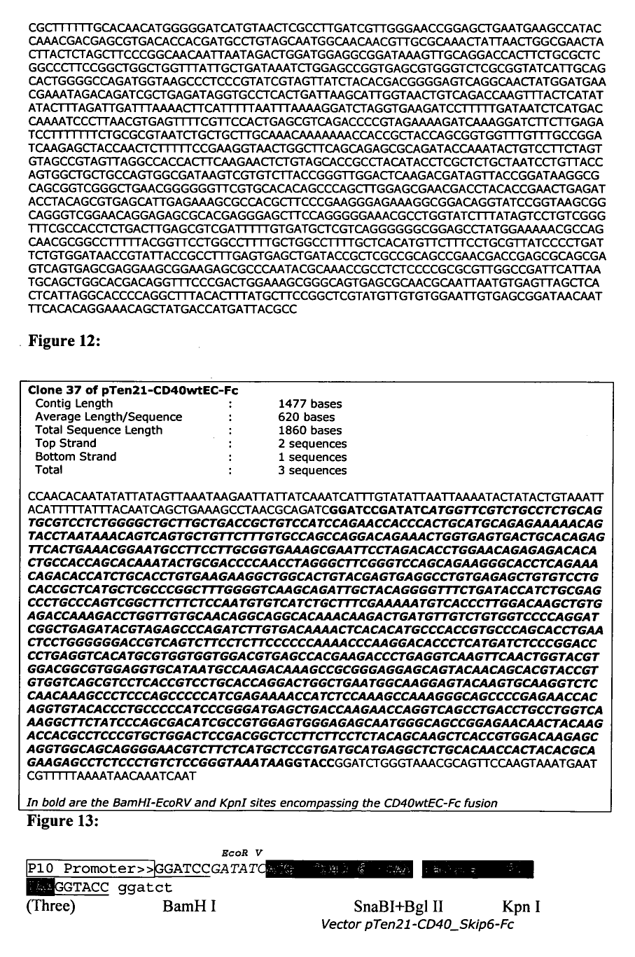

The sequence obtained for clone 37 is presented in SEQ ID NO: 19 and in FIG. 12, featuring the BamHI-EcoRV and KpnI sites marked in bold. The CD40 wtEC-Fc fusion sequences are shown in bold italics, and are separated by the TACGTA sequence.

4—Construction of pTen21-CD40_Skip6-Fc Vector:

Plasmid pET28a-CD40_{—}6 has been used to PCR-amplify a sequence extending from the internal StuI site to the end of CD40_Skip6 variant. The following primers have been used:

(SEQ ID 10)

5′-CACCATCTgCACCTgTgAAg-3′
(Stu40)

(SEQ ID: 20)

5′-CACAAgATCTgggCTCTACgTATTggTTggCTTCTTggCCA-3′
(Called 6Fc3′)

The amplified fragment of 330 bp was digested with StuI and BglII and inserted into the previously constructed pTen21-CD40 wtEC-Fc clone 37 vector cut with the same enzymes to obtain the vector presented in FIG. 13.

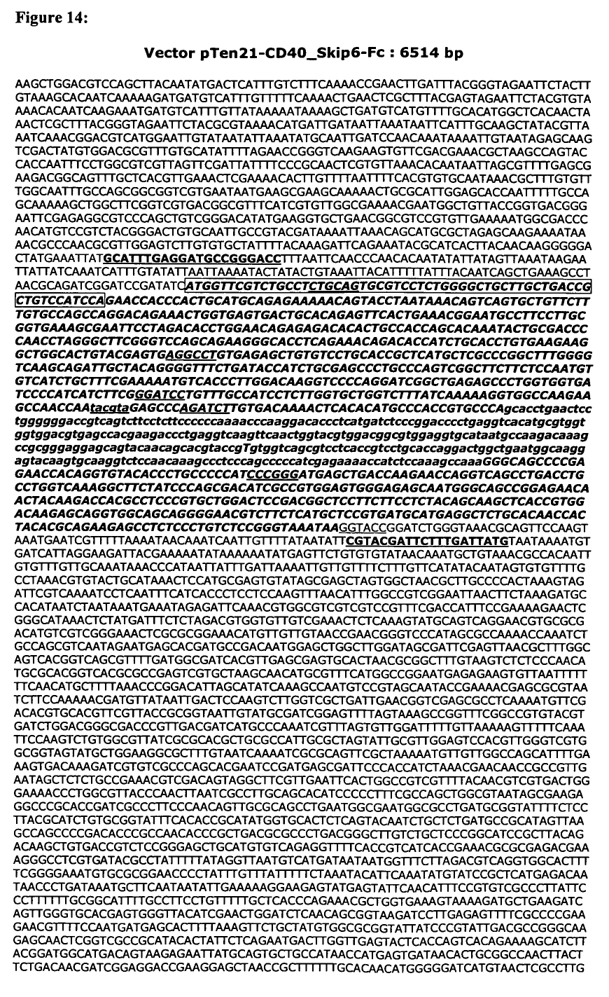

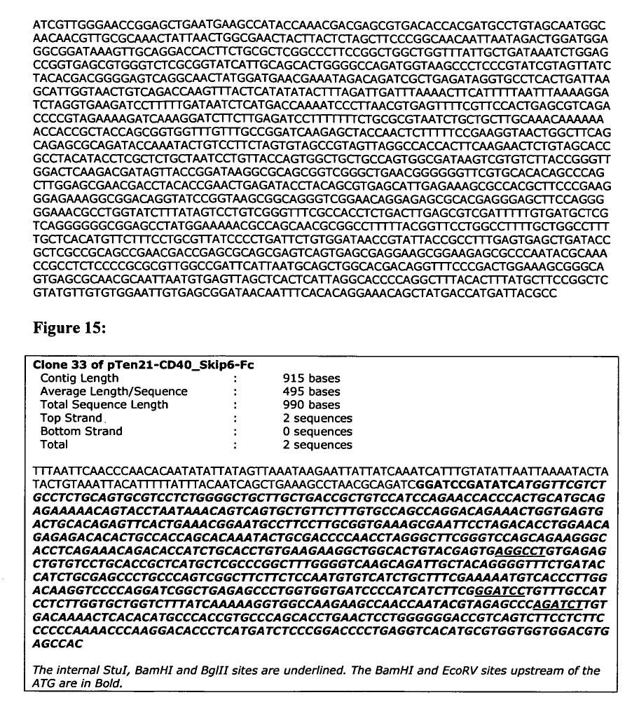

Selected plasmids have been sequenced with OQBT51 (SEQ ID NO:7) and the internal Stu40 (SEQ ID NO: 10) primer (to cover the region amplified by PCR before cloning, extending from the StuI to the BglII sites). Clone 33 presented 100% with the corresponding portion of the expected sequence of pTen21-CD40_Skip6-Fc vector given in SEQ ID NO: 21 and in FIG. 14, where primers are in bold and underlined, the CD40-skipping6-Fc fusion are shown in bold italics. Also shown is the polylinker and the signal peptide-encoding sequence. The sequence obtained for clone 33 is presented in SEQ ID NO: 22 and in FIG. 15, where the internal StuI, BamHI and BglII sites are underlined. The BamHI and EcoRV sites upstream of the ATG are in Bold. CD40-skipping6 sequences and the beginning of the Fc sequence are marked in bold italics.

### Example 3

### Protein Production and Processing in Baculovirus Protein Production and Processing from 1 L volume of Baculovirus

Baculovirus cells were transfected with the above constructs (BacTen-CD40 wtEC-Fc, BacTen-CD40_Skip6-Fc, BacTen-CD40 wtEC and BacTen-CD40_Skip6, corresponding to pTen21-CD40 wtEC, pTen21-CD40_Skip6, pTen21-CD40 wtEC-Fc and pTen21-CD40_Skip6-Fc, respectively), and cultured to produce the expressed protein. The baculoviral culture conditions are described in table 1 below. The initial cell density, the MOI used and the harvesting time in each experiment are indicated in table 1. Where indicated in Table 1, the anti-protease treatment was applied in cell culture medium at the following final concentrations: Pepstatin 10 μM, Leupeptin 2 μM, Pefabloc 1 mM. The final viability is indicated in Table 1 for each construct.

TABLE 1
Culture conditions

| | | | | Anti- | |
|---|---|---|---|---|---|
| | Initial cell | | Harvesting | Prote- | Final |
| Construct | density | MOI | Time | ases* | Viability |
| BacTen- | ˜800.000/ml | 2 | 72 hpi | − | 85% |
| CD40wtEC-Fc | | | | | |
| BacTen- | ˜800.000/ml | 2 | 72 hpi | + | 80% |
| CD40_Skip6-Fc | | | | | |
| BacTen- | ˜800.000/ml | 2 | 96 hpi | + | 92% |
| CD40wtEC | | | | | |
| BacTen- | ˜800.000/ml | 2 | 96 hpi | + | 75% |
| CD40_Skip6 | | | | | |

*Anti-proteases in cell culture medium at the following final concentrations: Pepstatin 10 μM, Leupeptin 2 μM, Pefabloc 1 mM.

Each collected 1 litre of supernatant was concentrated at 4° C. by tangential flow ultrafiltration in a 10 kDa cut-off Vivaflow 200 device (Vivascience cat# VF20PO). They were then dialysed in the same device against 50 mM Tris-HCl buffer pH 8.0. The final volume collected was around 25 ml. Solutions were then either frozen (non-fused constructs) or passed through a protein A column for purification, as described below in Example 4.

### Example 4

Purification of C-Terminal Fc-Tagged CGEN40 Variants Proteins:

The Fc-tagged proteins expressed using the baculovirus system were purified through a protein A column. The following reagents, resins and buffers were used:

Regents:

Albumin Standard {PIERCE, Cat # 23209}
Bradford reagent {BIO-RAD, Cat # 500006}
Citric acid monohydrate {MERCK, Cat # K91107244}
Dulbecoo's Phosphate Buffered Saline, concentrate X10 {Biological Industries, Cat # 020235A}
Simply Blue SafeStain {Invitrogen, Cat#LC6060}
Sodium Phosphate {Sigma, Cat # S7907}
Millipore filters, 0.22 [symbol] m (Cat# SCGP U11 RE)
Trizma base{Sigma, Cat # T1503}
   Resins:
Protein A Sepharose 4 Fast Flow {Amersham Pharmacia, Cat # 17097401}
(1st)
(2nd) Buffers:
Buffer A: 100 mM Tris HCl, pH7.5
Buffer B: 100 mM Citrate-Phosphate, pH3.5
Buffer C: 2M Tris
Buffer D: 1×PBS
   Total Protein Extraction:

Medium containing expressed protein and cells was centrifuged in 1500×g/10 min/4° C. using SLA-3000 Rotor, and the Supernatant was filtrated using 0.22 μm filter. Aliquots were stored at −70° C. To concentrate the samples the aliquots were defrosted in a water bath at RT, and then combined into a single vial. Then the sample was concentred by ten-fold, by using Vivaflow 10 kDa device using PES membrane, to the final volume of 100-150 ml. The device was washed with 30 ml medium from residual protein, and the wash was then added to the sample. Sample was stored at 4° C., until the purification step.

Purification:

Affinity Column—Protein A Sepharose

The pH of the protein sample was elevated to 7.0, using 1M Tris, pH7.5, and the sample was filtered using a 0.22 μm filter (approximately 5% of the final volume).

A 1 ml Protein A 5/5 Column, previously equilibrated with buffers B and A listed above, was loaded with the protein sample at 1 ml/min. The column was washed with buffer A—up to 80CV—until O.D280 nm was less then 0.01, followed by elution with buffer B. The pH of the eluted fractions was elevated with 1/10 volume of buffer C that was placed in the empty tubes before the elution step. The eluted fractions were subjected to SDS-PAGE, followed by Coumassie staining. Finally, the eluted fractions containing the protein were subjected to dialysis with 2×2 L buffer D.

Bradford Quantization of the Purified Protein.

Bradford quantization of the purified protein was carried out using cold Bradford reagent, diluted 1:5 in ddH₂O. BSA Standard commercial solution was made in the concentration range of 0.1 to 0.5 mg/ml. 200 μl of the diluted reagent and 10 μl of the standard/sample were added to microwell plates, in duplicates. The protein concentration was determined by comparison of the samples' O.D to the O.D of the known concentration of BSA.

Storage

The purified protein was stored in 1×PBS at −70° C.

### Example 5

### Recognition of the CD40-Skipping 6 Protein by Anti-CD40 Antibodies

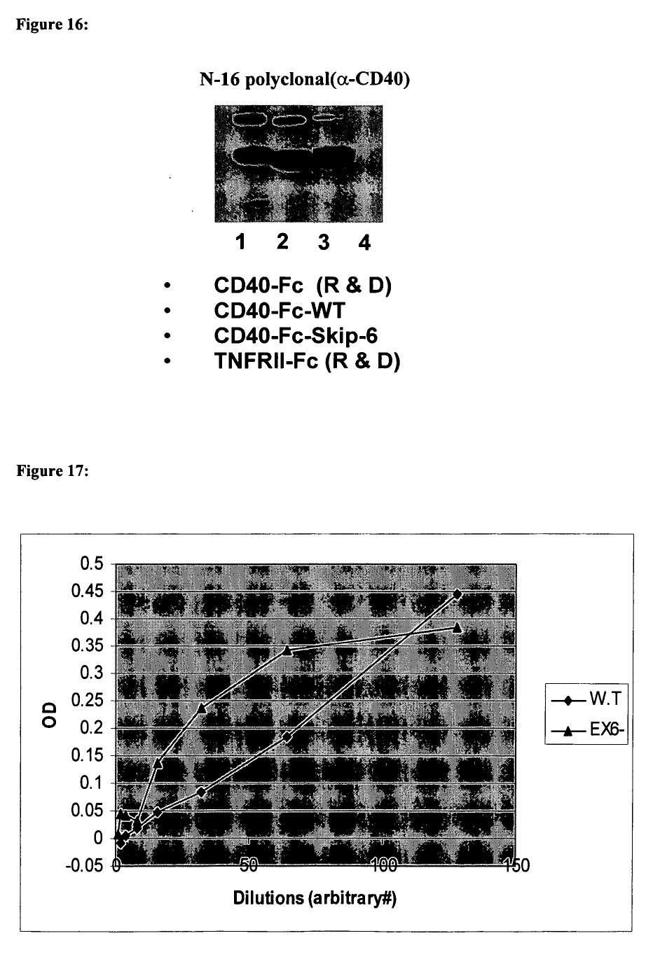

The CD40-skipping 6 protein was relatively unchanged by the purification, since it was easily recognised by a commercially available polyclonal antibody N-16 (polyclonal rabbit antibody from Santa Cruz (Cat num. Sc-974), which recognises the CD40 receptor, as can be seen from FIG. 16. FIG. 16 presents the results of Western blot analysis of the purified proteins as follows: line 1 presents CD-40 wtEC-Fc protein, line 2 presents CD-40 wt-Fc protein, line 3 presents CD-40 skipping6-Fc protein, and line 4 presents the TNFRII-Fc negative control. SDS-PAGE was performed as follows. The purified proteins were re-suspended in 30 μl 1×SDS-sample buffer containing 50 mM DTT (crude preparation). Following warming for 10 min and subsequent centrifugation, samples were loaded on Nu-PAGE gel buffer system (In-Vitrogen).

Following electrophoresis, for performing Western blots, gels were washed with cold transfer buffer for 15 min and taken for transfer to Nitrocellulose membranes for 60 min at 30 V using In-Vitrogen's transfer buffer and X-Cell II blot module. Following transfer, blots were blocked with TBS-5% skim milk (0.3% protein, 0.04% Tween-20) for at least 60 min. at room temperature or overnight at 4° C. Following blocking, blots were incubated with a commercially available N-16 antibody at ˜1 μg/ml for 1-3 hrs, washed with 0.05% Tween-20 in TBS, incubated with respective peroxidase-conjugated antibodies, washed with TBS-Tween-20 solution, followed by ECL. The results are shown in FIG. 16, demonstrating specific recognition between the N-16 antibodt and the various purified CD-40 proteins.

### Example 6

### Binding of the CD40-Skipping 6 Variant to the CD154

The binding capabilities of the purified skipping 6 protein Fc-fused, as compared to those of the wild type CD40-Fc fused protein, were assessed after the purification described above, by reacting these proteins with the CD154 ligand bound to 96 well ELISA plates, and quantifying the reaction using an ELISA reader.

Amine coupling of CD154 (ALX-522-015) to Nunc Peptide/Protein immobilizer plate was performed to prepare the ELISA plates. Binding analysis of Skip6 and WT CD40 was performed as follows.

CD154 protein was added to PBS buffer pH (7.2) to a final concentration of 0.2 ug/ml (total 10 ml) and the protein solution was added to 96 well ELISA plates (Maxisorp, Nunc) at concentration of 100 ul/well, 20 ng/well, and agitated at 4° C., O.N. (overnight). Following the incubation, the wells were washed three times with 300 ul/well PBST washing buffer (PBS-0.05% Tween20). CD40 proteins were added in serial dilutions of two fold each step (100 ul sample-diluent+100 ul sample in assay buffer (PBST+BSA), mixed by pipetting and followed by removal of 100 ul to the following well). Following the washing of the wells as above, the anti-CD40 antibody was added, diluted 1:2000 50 ul/well in assay buffer (PBST+BSA) and incubated for 2 hours at 25° C. (2 ul of stock material to total 4000 ul assay buffer). Then the plate was washed with PBST as above, and 100 ul/well of HRP-Avidin (Bender) were added 1:5000 in PBST+BSA, (1.5 ul to total 7.5 ml assay buffer). After an additional wash with PBST as above, the substrate was added (SSLO1+SSLO2, Bender) 100 ul/well, followed by incubation for 20 min. The reaction was stopped by addition of 100 ul stop solution (2N sulfuric acid) to each well, and the resultant absorbance was read at 450 nM, reference 620. The results of the ELISA binding of the CD40-Skipping 6 Protein to immobilized CD154 Ligand, as compared to the binding of the WT CD40, is presented in FIG. 17. The WT CD40 binding is represented by the line with diamonds, while the CD40-Skipping 6 binding is represented by the line with triangles.

As can be seen from FIG. 17, the purified CD40-skipping 6 protein retains its ability to bind CD154 ligand, and its binding profile is shown to be distinct from that of the wild type CD40 molecule. Skipping 6 is thus novel over the wild type molecule, exhibiting binding affinities and binding properties which differ from those of the wild type CD40 molecule.

### Example 7

### FACS Analysis of sCD40 Binding to Mouse Fibroblast Expressing Human CD154

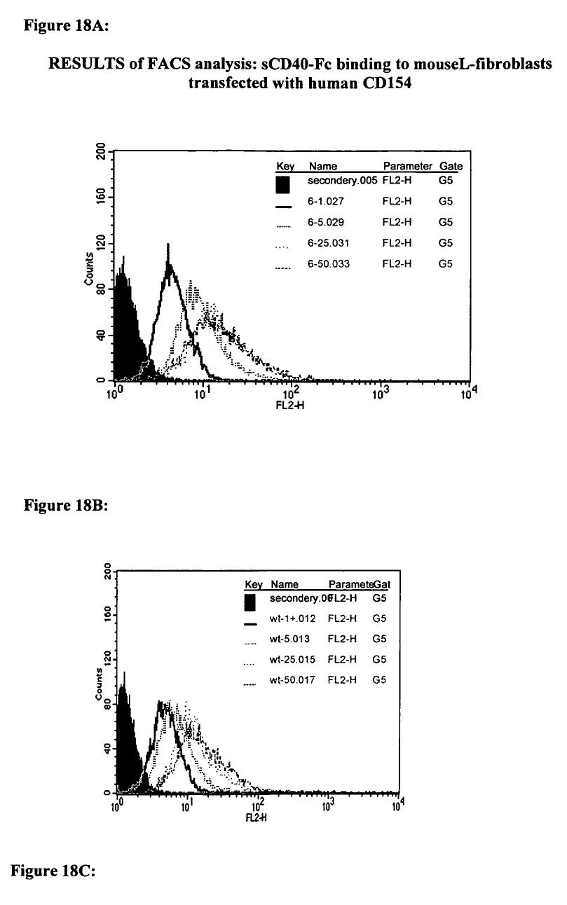

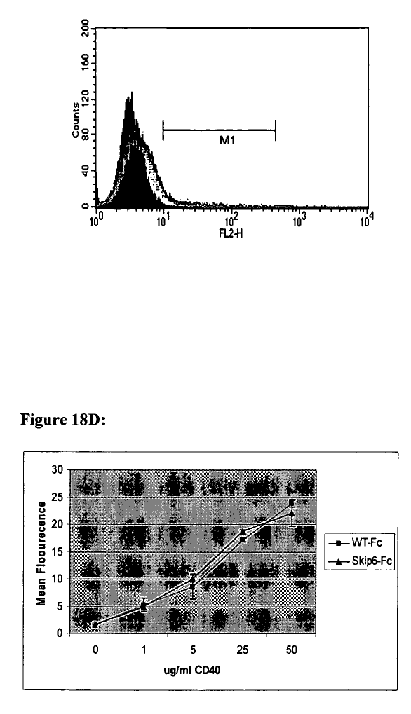

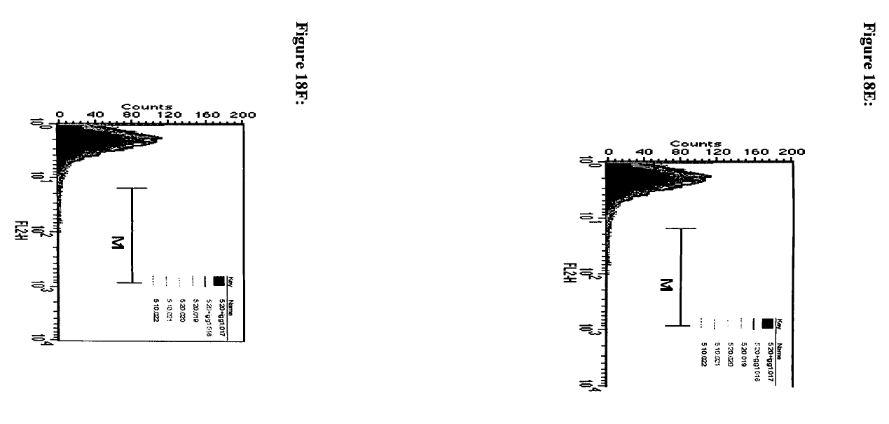

10⁶ mouse fibroblasts (stably transfected with full length human CD154) were incubated with either the known CD40 soluble variant, sCD40 WT (panel B, in FIG. 18), or CD40-skip6 (panel A, in FIG. 18). Both were Fc tagged, and present at a concentration of 1-50 μg/ml at 40 C for 60 min in total volume of 100 ul (0.5×10⁶ cells). As a control, mouse fibroblasts which do not express CD154 (panel C, in FIG. 18) were used. sCD40 binding was detected using PE-conjugated anti CD40 non-blocking antibody (clone EA-5). Analysis was performed using BD FACsCalibur. Panel D in FIG. 18 summarizes the mean fluorescence shift, as plotted versus various concentrations of CD40 protein.

Briefly, the FACS protocol was performed as follows. Mouse fibroblasts were trypsinized, and washed twice in PBS; cells were centrifuged at 1500 rpm for 10 min between washes. Next, the cells were re-suspended in FACS buffer (0.2% BSA and 0.02% sodium azide diluted 1/10 in PBS) to give 5-10×10⁶ cells mL. Cells were placed in FACS test-tubes at a volume of 100 or 200 microliters per tube.

Next, CD40 proteins were added (at concentrations of [1-50 μg/ml]), optionally with other treatments or controls, to the tubes containing the mouse fibroblast cells. The tubes were vortexed to mix and incubated for 1 hr at 4° C. in the dark. 5 ml PBS was added to each tube, after which the cells were pelleted to wash. The PBS buffer was removed by vacuum aspiration to reduce the volume back to 100 mL.

Next, 2 μL (1:50) anti CD40 non-blocking antibody (EA-5 mouse IgG1 PE-conjugated, obtained from Calbiochem) or controls (same isotype PE conjugated) were added to the tubes, and incubated for 30 min at 4° C. The process of washing was then repeated with 5 mL PBS. Next, 0.5 mL PBS was added to tubes, which were read in a FACS machine (BD FACSCalibur).

Alternatively after the second washing process, it is possible to perform fixation by adding 1 ml 4% paraformaldhyde to the cells and performing FACS analysis several days after the experiment. After fixation and before FACs analysis, the washing process should be repeated.

For this experiment, the controls included performing parallel assays with: mouse fibroblasts not expressing hCD154; purification mock; secondary Ab only and isotype control only (isotype refers to an antibody control, featuring the same type of antibody but one which is not able to bind CD40).

The axes are as follows: Y=cell counts; X=log scale of fluorescence intensity. M=A marker placed above the peak of positively stained cells on the histogram plot which provide the statistics of the stained population.

The results of the FACS analysis of the CD40 skipping 6 variants demonstrate significant binding to membrane CD154.

For the FACS assay without the Fc, the following procedure was performed (FIGS. 18E-F): 10⁶ mouse fibroblasts (stably transfected with full length human CD154) were incubated with sCD40 Wt and skip6 (figure E) non Fc at 40° C. for 60 min in total volume of 100 ul (0.5×10⁶ cells). As a control mouse fibroblasts which did not express CD154 were used (figure F). sCD40 binding was detected using PE-conjugated anti CD40 non-blocking antibody (clone EA-5). Analysis performed using BD FACsCalibur. Figure G summarized the mean fluorescence shift vs sCD40 concentrations. Significant binding was observed only with CD40-skipping 6 variant.

Panel G in FIG. 18 summarizes the mean florescence of the WT and the CD40 Skipping 6 proteins, when 10 or 20 ul were used in the reaction.

### Example 8

### Effect of the CD40 Variant on RANTES Secretion

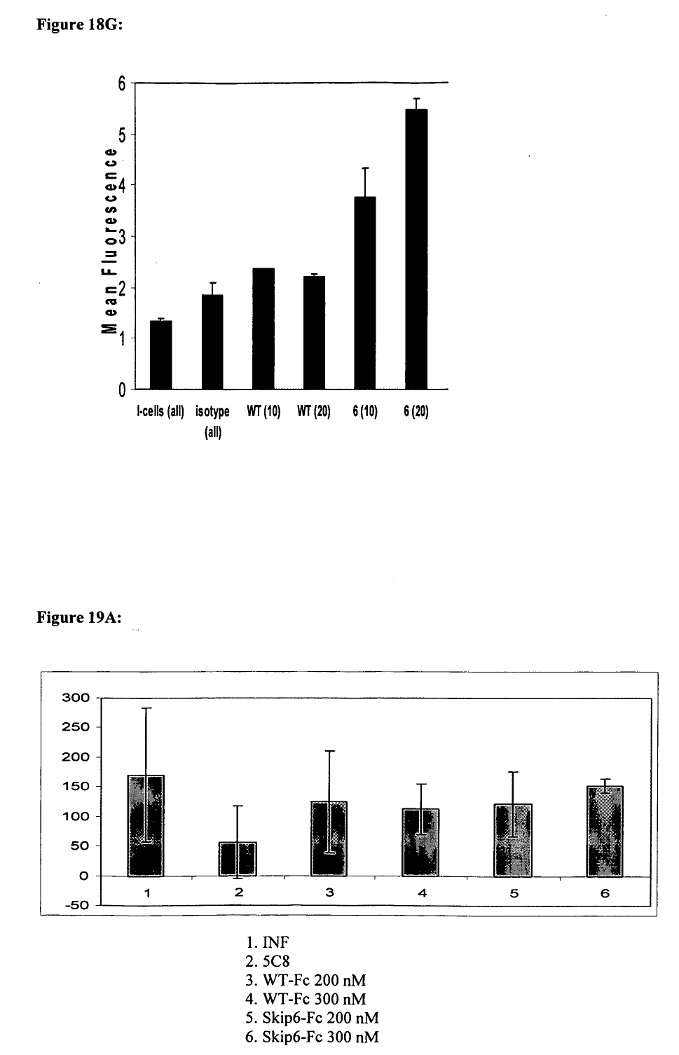

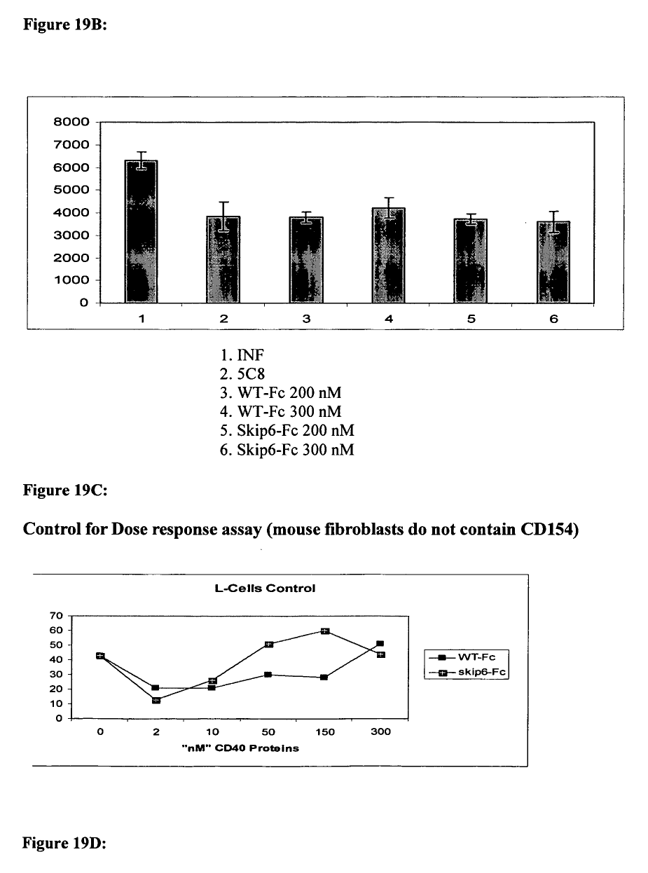

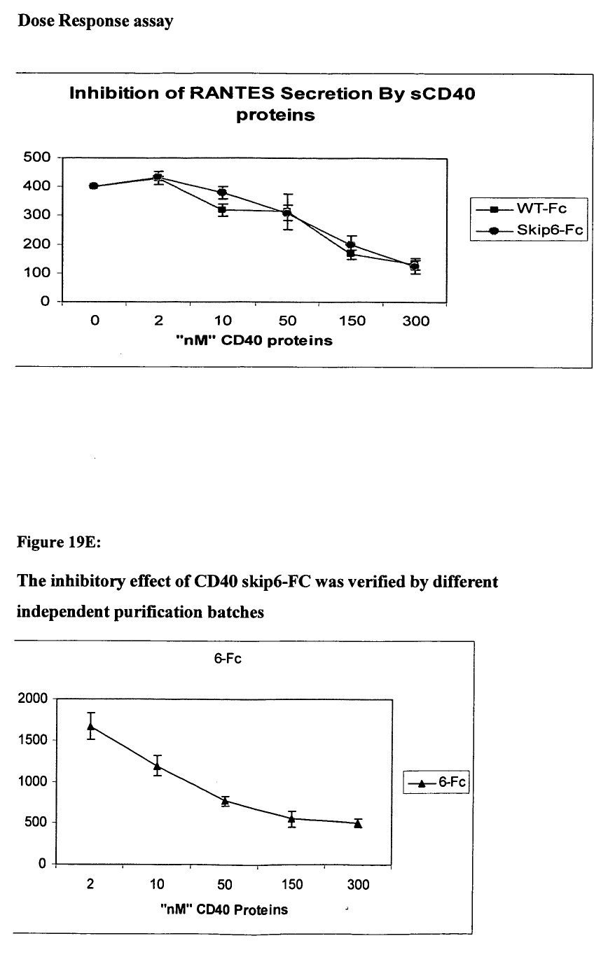

CD40-Skipping 6 protein was administered to a mixture of human peritoneal cells (HPMC cells), which express the CD40 receptor upon their membrane, and mouse fibroblasts transfected to express the CD154 ligand. The ability of the soluble skipping 6 protein, to compete with the CD40 membrane-bound receptor for binding to the secreted CD154 ligand, was thus tested. This ability was measured by determining the resultant level of the chemokine RANTES, which is a chemokine indicative of T cell activation, as compared to when a positive control of interferon (which raises the level of RANTES via a CD40-related pathway) was administered alone. The results are shown in FIGS. 19A-B.

RANTES Cell Assay Protocol

HPMC cells were grown in M199+10% FCS (Biological Industries, Bet Ha'emek, Israel), trypsinized (using trypsine from Biological Industries, Bet Ha'emek, Israel, 5 ml/75 cm² cell culture flask), and recultured into 96-well plates, to at least 80% confluence before further use.

Mouse fibroblasts/CD154-mouse fibroblasts were grown in DMEM+10% FCS (Biological Industries). Cells were trypsinized, pelleted (5 min. 500 at rpm), counted (½ vol cells+½ vol trypan blue) and resuspended in M199+10% FCS. Cells were then diluted (10,000 or 5,000 cells per ml).

CD40 proteins/antibodies were prepared in various concentrations in PBS according to the desired dose response/treatment, followed by adding the same volume of PBS to the negative and positive controls.

The CD40 protein variants were added to 100 mL of mouse fibroblasts or CD154-mouse fibroblasts in eppendorf tubes (final volume dependant on whether duplicates or triplicates were used) and incubated at R.T. (room temperature) for 1 h with rotation at 200 rpm for mixing.

During this incubation time the HPMC were prepared, by removing medium and washing cells twice, and adding 100 mL of fresh M199+10% FCS with or without 100 U/mL IFN (PeproTech, 50 U/μl).

The CD40 fibroblast cell mixtures were overlaid on the HPMC (110 ul/well) and incubated O.N (at 37° C., 5% CO₂).

100 mL were removed from the supernatant and placed into new 96 well plates, followed by diluting the samples 1/10 in M199+10% FCS and using diluted samples for the ELISA RANTES test.

The cells in the experiment plate were checked under the microscope. Optionally, the cell viability assay was performed.

The initial assay, the results of which are demonstrated in FIG. 19A, tests the control situation, in which the mouse fibroblasts used in conjunction with the HPMC cells were not transfected and thus did not express CD154. There were therefore no ligand present in the system, and Rantes production could not be activated via the CD40 pathway, such that its levels were relatively low. At left, interferon (INF) administration results in a Rantes level of approximately 150 pg/ml. An appropriate, commercially available antibody against the CD40 receptor lowered the level of Rantes to approximately 50 pg/ml. A soluble variant of the wild type CD40 receptor, was utilized, which is present as a chimera with the Fc (shown as “CD40-WT_FC”), but lacking the unique tail sequence of skipping 6. Administration of this wild-type protein at a concentration of either 200 or 300 nM, resulted in a Rantes level of approximately 100 pg/ml. Administration of the CD40-skipping 6 protein (shown as “CD40-skip6-Fc”), at either a concentration of 200 or 300 nM, gave results similar to the wild type protein.

Referring to the assay the results of which are demonstrated in FIG. 19B, the mouse fibroblasts used in conjunction with the HPMC cells, were transfected to express CD154. Both ligand and receptor were present, therefore, administration of the positive control interferon (INF) activated the CD40 pathway, and raised the Rantes level to 6000 pg/ml. Administration of the positive control antibody lowered this level to approximately 4000 pg/ml, as do both the soluble wild-type CD40 protein, and the CD40-skipping 6. Therefore, CD40-skipping 6 protein was able to compete with the membrane-bound CD40 receptor, present on the HPMC cells, for binding to the CD154 protein, and thus to interrupt CD40 ligand-receptor interactions.

Dose dependent inhibition of RANTES secretion by soluble CD40 proteins was tested and the results are presented in FIGS. 19C-D. As can be seen from the results of the dose response assay presented in FIG. 19D, where the CD154 ligand was present, the CD40-skipping 6 protein was shown to lower the level of Rantes in a dose-dependent manner, similar to the effect shown with the wild type secreted CD40 protein.

The inhibitory effect of the CD40-skipping 6 variant on RANTES secretion was verified by different independent purification batches, as demonstrated in FIG. 19E.

In the dose response control assay represented in FIG. 19C, the mouse fibroblast cells were untransfected and did not express CD154 ligand. Therefore, administration of the various soluble CD40 proteins had little influence on the level of Rantes.

### Example 9

### The Effect of the Combination of CD40 Variants and their Specific Antibodies on RANTES Secretion

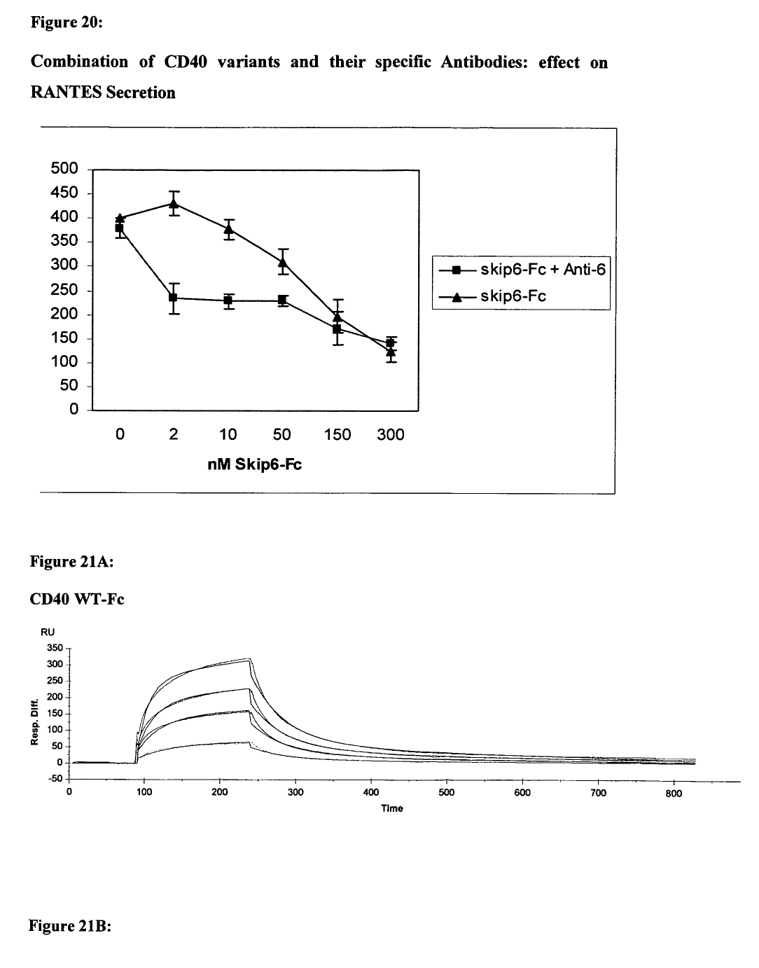

To test the effect of the combinations of CD40 variants and their specific Antibodies on RANTES Secretion, an antibody against the unique tail sequence of the CD40-skipping 6 variant was administered (at a fixed concentration of 10 ug/ml) to the HPMC and the transfected mouse fibroblast cells, along with the CD40-skipping 6 protein. The antibody does not recognize the wild type CD40 protein. Surprisingly, when the antibody was present in addition to the CD40-skipping 6 protein, the Rantes level was lowered to a level of approximately 240 pg/ml (as seen at a concentration of 2 nM of CD40-skipping 6 protein), as demonstrated in FIG. 20. The presence of the antibody thus acts to further inhibit the CD40 pathway, and instead of binding to and inactivating the CD40-skipping 6 protein, it apparently stabilizes it or enhances its ability to bind CD154 ligand. This indicates potential therapeutic use of the CD40-skipping 6 protein in combination with a specific anti-CD40-skipping 6 antibody, in order to interrupt CD40 ligand-receptor interactions.

### Example 10

### CD40 WT-Fc and CD40-Skip6-Fc Binding to CD154 Using Biacore

A Biacore system was utilized to measure the binding properties of the CD40-skipping 6 protein to CD154 (the CD154 ligand was bound as a secondary protein layer upon the Biacore chip). (Fivash, M., et al. Current opinion in Biotechnology, 1998, 9:97-101).

A Biacore CM5 chip was activated with NHS/EDC. Anti CD154 antibody (Enhancer) was coupled to the chip (120 ul of 100 ug/ml in 10 mM NaAc pH4 injected at 10 ul/min) to give 10K-15 K Ru (resonance units), and the reaction was stopped by washing with 10 ul 1M ethanolamine. In the second stage rsCD154 was injected (60 ul of 10 ug/ml in 10/ul min) to give around 1000Ru above the baseline established by the enhancer. This interaction was very stable and showed minimal leakage of CD154. As a control for the specificity of binding two more surfaces were built, the first of which was only activated (NHS/EDC) and suppressed (ethanolamine) as a control, and the second of which was only activated with the coupled anti CD154 antibody without CD154. At the next stage serial dilutions of CD40-Fc tagged proteins were injected over the chip surfaces, the results shown in the FIGS. 21A-B are representation of the Ru response of the surface containing the CD154 minus the response on the control surfaces.

Referring to FIGS. 21A and B below, the graphs indicate the association and dissociation profiles for various concentrations of the CD40 proteins, over the time-course of the experiment. Time is plotted on the X axis, with each concentration of CD40 protein represented by a separate plotted line raised one above the other. The CD40 wild type or the CD40-skipping 6 protein were injected at time=100, and binds to the CD154 upon the chip until time=280. Both the CD40-skipping 6 (FIG. 21B) and the wild type protein (FIG. 21A) were found to dissociate from the chip at approximately time=250, with the slope of the dissociation curve being similar in both skipping 6 and the wild type. Thus both have similar avidity for the CD154 ligand.

FIGS. 21A-B represent Biacore CM5 chip; Ligand: rsCD154+antiCD154 Ab (Enhancer); Analytes: CD40 WT-Fc (A) Skip6-Fc (B) (20-400 nM); Data analysis: Biaevaluation software using langmuir 1:1 binding model (A+B=AB).

### Example 11

### mRNA Expression of the CD40 Variant

The following experiment was performed to determine the mRNA expression levels of native CD40-skipping 6 protein, as compared to wild type CD40, in a K562 erythroleukemia cell line.

Identification of the CD40-skipping6 cDNA was done from RT-PCR of the K562 cell line. The K562 cell line was thawed and grown for 3 days in 40 ml. RNA was prepared from 30 ml cells. RNA conc. 0.9 ug/ml. Dnase treatment was done using the Ambion kit. RT-PCR was done with oligo dt using 2 ug of superscript in 20 ul at 42 C for 1 h, than 70 C for 15 min.

PCR was done using 2 ul of this reaction. PCR conditions: annealing 62 C/45 sec elongation/35 cycles. Four different fragments were purified from the 1.2% agarose gel and sequenced.

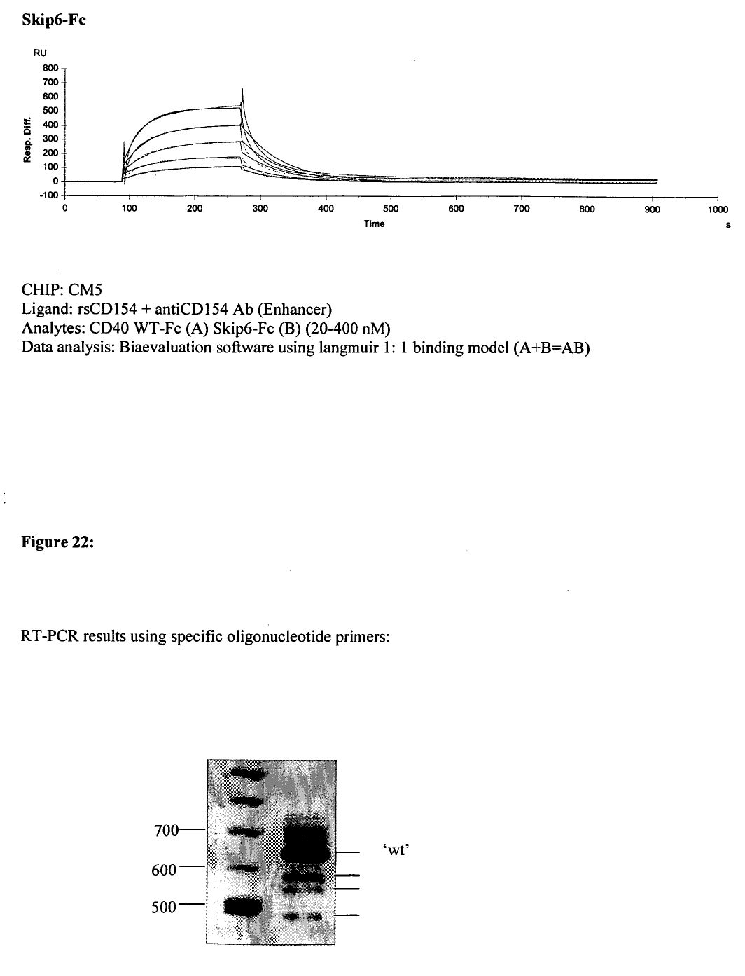

As demonstrated in FIG. 22, two fragments of 500 bp and 550 bp were shown to be other splice variants of CD40, while a 600 bp fragment was found to be CD40-skipping 6, and a 650 bp fragment was found to be CD40 wild type.

Results Expression of (CD40 Variant in K562 Cell Line

The expression of different CD40 splice variants was detected in K562 erythroleukemia cell line, and is presented in FIG. 22. The secreted CD40 splice variant which is the issue of the current patent application is “skipping exon 6 variant”, and is addressed in FIG. 22 as “exon 6”. “wt” represents the original “wild type” form of the molecule. The “exon (5+6)” is a membrane form of CD40 splice variant, addressed also in the attached PNAS paper (Tone, M., et al., 2001, PNAS 98:1751-1756).

### Example 12

### Functional Analysis of the CD40 Splice Variants in K562 Cells

It is well accepted that CD40 mediates antiapoptotic and proliferative signaling for normal resting B cells (Tsubata, T, et al, 1993, Nature 384: 645-648). In contrast CD40 ligation in carcinoma cell lines results in growth inhibition and sensitizes these cells to apoptosis induced by a variety of agents, including TNF-, anti-Fas, and cytotoxic drugs (Eliopoulos, A. G., Oncogene 13:2243-2254). Furthermore, when exogenously expressed, CD40 has been shown to transduce apoptotic signals in certain cell lines of epithelial or mesenchymal origin. Due to the involvement of CD40 in apoptosis the alteration of the expression pattern of the different variants of CD40 was tested as a response to apoptosis in K562 cells. Also the expression pattern of CD40-skipping 6 variant was compared to that of the wild type CD40.

Samples of RT reactions of K562 treated with etoposide were prepared and used for PCR using CD40 primers (35 cycles). Etoposide (Sigma) is known as double-stranded DNA breakage and apoptosis inducing agent. The RT reactions were checked before the analysis to exclude possible genomic contamination and to ensure similar cDNA concentrations in the different samples, using quantitation with GAPDH (not shown).

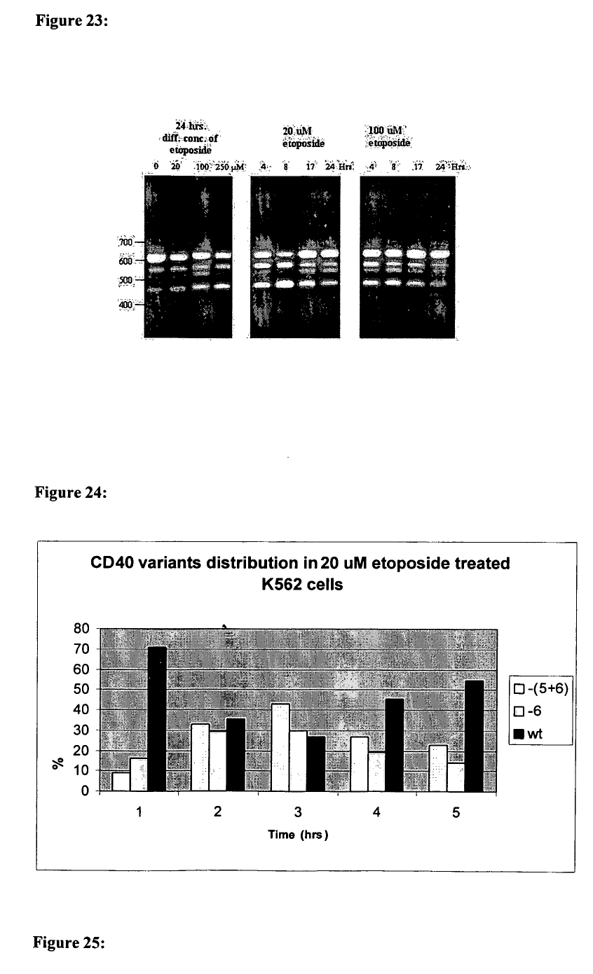

The results are shown in FIG. 23.

The bands resulting from the RT PCR reactions, demonstrated in FIG. 23, were quantitated, and FIG. 24 presents the percentage of the expression of each splice form out of the total CD40 expression levels for K562 cells treated with 20 micromolar Etoposide for various time intervals.

As can be seen from the results, while the original “wild type” CD40 molecule's expression levels decrease upon double-stranded DNA breakage induced by Etoposide, the expression levels of the secreted CD40 splice variant skipping exon 6 (“skipping 6”) increase. The optimal effect is observed at 8 hours of treatment of K562 cells with 20 uM Etoposide. The CD40-skipping 6 mRNA transcript therefore has a physiological expression pattern which is different from that of the wild type CD40 receptor protein, when apoptosis is induced in erythroleukemic cells.

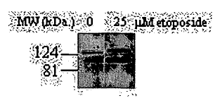

Since K562 cells do not have an active p53, it was decided to test whether these cells still enter apoptosis after treatment with etoposide in the relevant time frame. For this purpose K562 cells were treated with 25 pM etoposide for 8 hours and the activation of caspases, one of the hallmarks of apoptosis, was measured. To measure caspase activation, the cells were lysed, immunobloted and PARP, a known substrate for caspase-3, was probed using anti cleaved PARP antibody (Cell Signaling, Beverly, Mass.). The results are shown in FIG. 25, and they demonstrate the appearance of a band in the expected size in the treated cells, thus indicating that the effect seen by etoposide may be part of the apoptotic machinery.

The effect on the expression of the secreted splice form compared to the expression of the wild type form in the course of apoptosis can be explained by the dominant negative nature of the secreted form. While the original antiapoptotic CD40 is repressed during DNA damage and subsequent apoptosis, the skipping 6 secreted form might compete with the wild type form by binding to the ligand without subsequent signaling. Therefore, the secreted molecule, which is overexpressed upon DNA damage, might act as an antagonist of the original CD40 molecule and can be utilized accordingly, to disrupt the CD40 receptor-ligand interaction.

### Example 13

### Treatment of Atherosclerosis by Administering CD40-Skipping 6 Splice Variant Protein

A human subject diagnosed with atherosclerosis is treated with a CD40-skipping 6 splice variant protein to reduce the symptoms associated with the disease. A CD40-skipping 6 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. Additional doses are administered as warranted from about daily to about weekly.

### Example 14

### Treatment of Cancer by Administering CD40-Skipping 6 Splice Variant Protein

A subject diagnosed with colorectal cancer is treated with a CD40-skipping 6 splice variant protein to reduce the symptoms associated with the disease. A CD40-skipping 6 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms and response of the cancer to treatment. Depending on the physical characteristics, additional doses are monitored from about daily to about weekly.

### Example 15

### Treatment of a Chronic Inflammatory Disease by Administering CD40-skipping 6 Splice Variant Protein

A subject diagnosed with inflammatory bowel syndrome is treated with a CD40-skipping 6 splice variant protein to reduce the symptoms associated with the disease. A CD40-skipping 6 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms. Depending on the physical characteristics, additional doses are monitored from about daily to about weekly.

### Example 16

### Treatment of Atherosclerosis by Gene Therapy with CD40-Skipping 6 Splice Variant

A subject diagnosed with atherosclerosis is treated by administering a gene therapy construct capable of expressing a CD40-skipping 6 splice variant protein to reduce the symptoms associated with the disease. The CD40-skipping 6 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 17

### Treatment of Cancer by Gene Therapy with CD40-Skipping 6 Splice Variant

A subject diagnosed with cancer is treated by administering a gene therapy construct capable of expressing a CD40-skipping 6 splice variant protein to reduce the symptoms associated with the disease. The CD40-skipping 6 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 18

### Treatment of Chronic Inflammatory Disease by Gene Therapy with CD40-Skipping 6 Splice Variant

A subject diagnosed with chronic inflammatory disease is treated by administering a gene therapy construct capable of expressing a CD40-skipping 6 splice variant protein to reduce the symptoms associated with the disease. The CD40-skipping 6 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 19

### Immune Rejection of Transplanted Organs; Treatment with CD40 Splice Variants

Experimental Protocol—Assessment of Treatment with CD40 Variant on Heart Transplant Rejection

The ability of CD40 variant according to the present invention to block or at least attenuate rejection of heart transplant allografts is examined according to the “two heart” transplantation model. This model was previously described in Mannon et al, Journal of Heart and Lung Transplantation, vol 18, no 9, pp 819-827, 1999 and Corry et al, Transplantation, vol 16, no 4, pp 343-350, 1973, both of which are hereby incorporated by reference as if fully set forth herein.

Briefly, mice are transplanted with a second heart, so that the effect of any treatment protocol on transplantation efficacy can be separated from the effect of rejection on heart function and overall physiology of the mice. Preferably, a first group of mice receives no treatment according to the present invention for preventing transplant rejection; a second group of mice receives the treatment only after transplantation of the second heart; a third group receives the treatment both prophylactically and after transplantation; a fourth group receives the treatment but no operation; and the fifth group receives a “mock” operation (in which all steps are performed apart from inserting the second heart into the mice, while overall timing is observed for the mock operation). The mice are then monitored for both the function of the second heart (if present) and also the overall health of the mice, including measurements of various parameters concerning the effect of the treatment on the mice and also on the second heart. A more detailed description is provided below, divided into sections. The first section, “Treatment Protocol”, describes the protocol for administering the treatment according to the present invention to the animals, and also for monitoring its efficacy. The second section, “Surgical Protocol”, describes the surgical protocol used on the animals.

Treatment Protocol

Mice are injected with either CD40 variant according to the present invention (preferably the “skipping 6” variant) in groups 2, 3 and 4, or with sterile saline, for groups 1 and 5. Preferably the amount of CD40 injected is 10-500 micrograms per mouse and more preferably is about 250 micrograms per mouse. The CD40 variant is placed in a sterile aqueous solution, optionally with a suitable buffer and is injected i.p. into the mice once per day.

Mice are examined on a daily basis for the following parameters: primary heart function (ie the function of the mouse's original heart), secondary heart function (if present) and optionally one or more serum markers. As non-limiting examples only, FITC labeled T11, B1 (Coulter), and FN18 monoclonal antibodies may optionally be used to assess the percentage of CD2 (T cell/NK cell), CD20 (B cell), and CD3 (T cell) positive cells respectively. Heart function is monitored by measuring heart beat rate.

After 30 days (or at the death of the mouse), the mice are sacrificed and the hearts (both primary and secondary) are removed. Tissue samples are fixed for histopathology as described in Mannon et al or Corry et al, by being placed in formalin and stained with hematoxylin-eosin. Other samples are fixed for morphological examination. In addition, before samples are taken, the overall heart morphology is examined. Examinations of the heart or of tissue samples are performed by a pathologist who is masked to the experimental groups. Sections (and the overall heart) are graded with regard to the severity of histopathological abnormalities, for example according to a scale described in a second reference by Mannon et al (Transplantation 1995; vol 59: pages 746-755, incorporated by reference as if fully set forth herein), which is stated to include consideration of the following parameters: vascular changes, myocyte injury and interstitial infiltrate. Optionally, an overall score may be obtained by combining measurements of these parameters.

Mice receiving the treatment with the CD40 variant according to the present invention, with or without surgery, are found to tolerate the treatment well. Function of the second heart in mice that also received the CD40 variant is found to be extended, and pulsatile action of the second heart is found to still be present at least one day longer for mice receiving the CD40 variant than for those mice that do not receive the CD40 variant. Also, the presence of histopathological abnormalities is found to be much lower in the second heart of mice receiving the CD40 variant, with fewer gross morphological abnormalities, than in the second heart of mice that do not receive the CD40 variant.

Surgical Protocol

16 mice receive a second heart, transplanted as an allograft into the chest cavity, according to the following protocol. Donor mice of a MHC disparate strain are selected so as to be of a tissue type that will be rejected by the recipient mice (for example, hearts from [BALB/C×DBA/2J] F1 mice (which can be purchased from Jackson Laboratory, Bar Harbor, Me., USA) can be inserted into C57BL/6 mice). 16 recipient mice and 16 donor mice are prepared for surgery, including anesthetizing mice with a suitable small animal anesthetic (such as chloral hydrate administered i.p. at a single dose of 0.1 ml of a 3.6% solution per 10 g of body weight). The anesthetized mice are shaved in the area of the chest, which is then cleaned with a disinfectant such as 70% alcohol.

The recipient mice are prepared as follows (and are preferably prepared before the donor mice). The chest (abdominal) area is magnified for a clear view of the blood vessels, and the chest/abdominal area is opened. At least 2 mm of the aorta and vena cava blood vessels are dissected free of surrounding tissue below the renal vessels. Preferably only one lumbar artery and vein are ligated to provide the portions of these blood vessels that will be connected to the corresponding blood vessels on the donor heart. Proximal and distal ties of surgical thread are placed around the aorta and vena cava, to prepare for later occlusion of these vessels. A gauze pad or other sterile material soaked in a sterile liquid, preferably saline, is placed in the abdominal cavity.

The donor mice are prepared as follows. The chest/abdominal area of the mouse is opened and a heparin solution is injected into the inferior vena cava. The heart is removed as rapidly as possible, preferably within 3-4 minutes. The inferior vena cava is ligated with surgical thread and divided below (inferior to) the tie, while the superior vena cava is ligated with surgical thread and divided above the tie. The aorta and pulmonary arteries are separated and divided. Excess blood is gently massaged from the heart. The pulmonary veins are then ligated and the heart is removed.

The ties around the aorta and vena of the recipient mouse are tightened and incisions are made in the recipient blood vessels in preparation for being joined to the corresponding donor heart blood vessels. As this process is being performed, the donor heart is preferably bathed in chilled saline or Ringer's lactate solution to prevent damage from ischemia. After the blood vessels are joined, the inferior vascular tie is released first, to fill the inferior vena cava and the pulmonary artery from the donated heart with blood. The heart is preferably also externally warmed, for example by being bathed in warm Ringer's lactate solution. If necessary, cardiac massage is performed to start the heart beating.

The mock operation is performed similarly, including with regard to timing, except that a second heart is not inserted into the mice undergoing the mock operation.

Experimental Protocol—Renal Transplant

A preferred, exemplary model system for testing efficacy of CD40 splice variants for transplant efficacy is the primate renal allograft model disclosed in published U.S. Patent Application No. 20020119150 and in Kirk et al. (1997), 94 Proc. Natl. Acad. Sci. USA 8789-8794, the teachings of both which are incorporated by reference herein. The present rhesus monkey model has been shown repeatedly to be a rigorous test of immune manipulation: one that is exquisitely sensitive to even minor changes in allograft function or adverse effects on recipient wound healing and immune system function. In addition, it has biological similarity to human renal transplantation. Specifically, genes that encode MHC proteins are well conserved between rhesus monkeys and humans, and their rejection of vascularized organs closely parallels that seen clinically.

Materials and Methods

CD40 splice variant is prepared as described herein.

MHC Typing and Donor/Recipient Selection

Donor-recipient combinations and animals chosen for third party cells are selected based on genetic non-identity at both MHC class I and class II. Class I disparity is established by one-dimensional isoelectric focusing. Class H disparity is established based on the results of unidirectional mixed lymphocyte reactions (MLRs). In addition, the animal's DRB loci are preferably verified to be disparate by denaturing gradient gel electrophoresis and direct sequencing of the second exon of DRB. Vigorous T cell responsiveness of the recipient towards the donor is confirmed in vitro for all donor-recipient pairs. The experiments described in this study are conducted according to the principles set forth in the “Guide for the Care and Use of Laboratory Animals” Institute of Laboratory Animals Resources, National Research Council, DHHS, Pub. No. N1H) 86-23(1985).

In Vitro Cellular Analysis

Unidirectional MLRs are performed on all animals prior to transplantation and on rejection free survivors after 100 days. Each animal is tested against all potential donors to establish the highest responder pairs for transplantation. Responder cells are incubated with irradiated stimulator cells at 37 C for 5 days. Cells are pulse-labeled with 3H-thymidine and proliferation is monitored by 3H-thymidine incorporation. Polyclonal stimulation with Concanavilin A (25 mcg/ml) is a positive control. A stimulation index is calculated by normalization to self reactivity. For in vitro dose response studies, CTLA4-Ig or humanized 5c8 may optionally be added to the MLR (ie added as a treatment with CD40 splice variant according to the present invention) on day 1 at concentrations ranging from 100 mcg/ml to 0.01 mcg/ml. Combined treatments are performed by varying the CTLA4-Ig concentration and the humanized 5c8 concentration, and holding the CD40 splice variant concentration steady at 50 mcg/ml.

Peripheral blood lymphocyte phenotype analysis is performed prior to transplantation and periodically during and after drug therapy. Assays preferably include 0.2 ml of heparinized whole blood diluted with phosphate buffered saline and 1% fetal calf serum. FITC labeled T11, B1 (Coulter), and FN18 monoclonal antibodies may optionally be used to assess the percentage of CD2 (T cell/NK cell), CD20 (B cell), and CD3 (T cell) positive cells respectively. Red blood cells are removed from the preparation by ACK lysis buffer (0.15 M NH4Cl, 1.0 mM KHCO3, 0.1 mM Na2EDTA, pH 7.3) treatment following staining. Cells are subjected to flow cytometry immediately, or following fixation in 1% paraformaldehyde. Flow cytometry is performed using a Becton Dickinson FACSCAN.

Renal allografts are a well studied model. Renal allotransplantation is preferably performed as follows. Outbred juvenile (1 to 3 years of age) rhesus monkeys, seronegative for simian immunodeficiency virus, simian retrovirus, and herpes B virus, may optionally be obtained from the Primate Center (University of Wisconsin) or LABS (Yemassee, S.C.). Procedures are performed under general anesthesia using ketamine (1 mg/kg, i.m.), xylazine (1 mg/kg, i.m.) and halothane (1%, inhaled). Transplantation is performed between genetically distinct donor-recipient pairs, preferably as determined by the MHC analysis described above. The animals are heparinized during organ harvest and implantation (100 units/kg). The allograft is implanted using standard microvascular techniques to create an end to side anastamosis between the donor renal artery and recipient distal aorta as well as the donor renal vein and recipient vena cava. A primary ureteroneocystostomy is then created. Bilateral native nephrectomy is completed prior to closure.

Animals are treated with intravenous fluid for approximately 36 hours until oral intake is adequate. Trimethaprim-sulfa is administered for 3 days for surgical antibiotic prophylaxis. Each animal receives 81 mg of aspirin on the day of surgery. The need for analgesia is assessed frequently and analgesia may be maintained with intramuscular butorphanol. Animals are weighed weekly. Skin sutures are removed after 7 to 10 days. CD40 splice variant according to the present invention, optionally with CTLA4-Ig and/or humanized 5c8, are given intravenously at doses and dosing schedules varying based on accumulating experience with the agents. No other immunopharmaceuticals are administered. Serum creatinine, and whole blood electrolytes (Na+, K+, Ca2+) and hemoglobin are determined every other day until stable and then weekly.

Pathological Analysis

Biopsies are performed on animals suspected of having rejection using a 20-gauge needle core device (Biopty-Cut, Bard). Standard staining with hematoxylin and eosin is performed on frozen or formalin fixed tissue to confirm the diagnosis of rejection. Animals are sacrificed to determine the complete extent of rejection (or lack thereof).

Experimental Protocol—Skin Transplant

The Primate Skin Allograft Model System is a very demanding model for transplant rejection and for determining the efficacy of a particular treatment in blocking such rejection. The model is demanding because the skin is a notoriously difficult tissue with which to achieve/maintain engraftment. Autografts are not always possible and there is therefore currently a need for skin allografts and xenografts, for example for burn victims and/or those requiring reconstructive surgery for birth defects or other conditions, patients suffering traumatic injuries (e.g., partial or complete amputation of a limb or other body parts) and those who need plastic surgery, as well as many other potential applications of skin grafts. This model may optionally be assessed in primates as described below.

Nine primates (rhesus macaques) are used in the pre-clinical studies. The recipient animals are allogeneic to the donor animals. Graft donor/recipient pairs are assigned based on MLR high response and class II disparity determined by PCR analysis.

On the day of transplantation (day 0), preferably abdominal skin (full thickness) is taken from donor animals and defatted in normal saline with scissors and #10 blade. Abdominal skin wounds on the recipient animals are cleaned, and then ellipses of recipient skin are taken from the back at transverse axillary line. Both procedures are performed using aseptic technique. Skin grafts are placed on left scapula for autografts and right scapula for allografts.

CD40 splice variant is administered intravenously in a suitable amount (for example 20 mg/kg) to each recipient prior to grafting. Additionally, CD40 splice variant (for example 10 mgs per animal) may optionally and preferably be administered beneath the graft by injection into each recipient's graft bed at the time of grafting.

Induction and maintenance therapy preferably features CD40 splice variant be administered at a suitable amount (for example 20 mg/kg) is given in a suitable dosing schedule (for example, on days 0, 3, 10, 18, 28 and then monthly).

It should be noted that this monotherapeutic regimen may optionally be augment with one or more additional therapeutic agents, such as conventional drugs for preventing or treating transplant rejection, a non-limiting example of which is cyclosporine. Other non-limiting examples include rapamycin and/or biologicals such as CTLA4-Ig, which is an extracellular protein fused to Fc. Preferably the CD40 splice variant according to the present invention is also fused to Fc. Without wishing to be limited by a single hypothesis or to a single effect, combination therapy may optionally offer such benefits as permitting efficacy to be achieved with reduced amounts of immunosuppressants and/or reduced dosing frequency, and/or more focused blockade of the immune system (rather than the current treatment models, which essentially shut down the entire immune system and which therefore have serious side effects).

Skin grafts are preferably examined on day 1 post-transplantation and daily thereafter.

In ongoing transplant studies, donor specific transfusion (DST) has been utilized generally according to techniques described in U.S. Pat. No. 5,683,693. Administration of donor antigen (e.g. whole blood) with the CD40 splice variant may further reduce the incidence of graft rejection.

Assessment of Treatment with CD40 Variant an Transplantation of Other Types of Organs

The effect of CD40 variant according to the present invention on the amelioration of transplant rejection for other organs is optionally assessed. For example, renal transplantation into non-human primates as a model for the assessment of treatment efficacy is described in Elster et al, Transplant Immunology, vol 13, 2004, pp 87-99. Non-human primates share many surface antigens with humans, and also have highly similar physiology, making them an important model for organ transplant. Various parameters to be considered for rejection of renal organ transplants are described in the article by M. Kamoun in Clinical Biochemistry, vol 34, 2001, pp. 29-34. These parameters include measurement of mRNA expression of such genes as those responsible for cytotoxic attack molecules and various cytokines, such as IL-2, IL-4, IL-10 and TGF-beta. Such measurement may optionally be performed with RT-PCR, for example, as is well known in the art.

Although the invention has been described with reference to specific methods and embodiments, it is appreciated that various modifications and changes may be made without departing from the invention.

The descriptions given are intended to exemplify, but not limit, the scope of the invention. Additional embodiments are within the claims.

1. A pharmaceutical composition comprising a pharmaceutically acceptable carrier, and as an active ingredient an agent comprising the amino acid sequence selected from the group consisting of:
(i) the amino acid sequence depicted in SEQ. ID No. 1;
(ii) a fragment of at least 10 amino acids of the amino acid sequence of (i), having at least four consecutive amino acids of the segment 166-203 of SEQ. ID No. 1, said fragment still having CD40-L binding properties substantially as those of the sequence of (i);
(iii) a variant of the amino acid sequence of (i) or (ii) wherein up to 20% of the amino acids have been replaced, chemically modified or deleted, wherein the sequence substantially maintains the CD40-L binding properties of (i); and
(iv) a chimeric protein comprising the amino acid sequence of (i), (ii) or (iii), conjugated to another entity.

2. A pharmaceutical composition according to claim 1, wherein the fragment includes the at least 4 consecutive amino acids of 166-203 attached to at to a sub-sequence comprising the at least one of the following amino acids E74, Y82, N86, D84, E114, E117 of the CD40.

3. A pharmaceutical composition according to claim 1, wherein up to 15% of the amino acids have been replaced, chemically modified or deleted, wherein the sequence maintains the CD40-L binding properties of the sequence specified in (i).

4. A pharmaceutical composition according to claim 1, wherein up to 10% of the amino acids have been replaced, chemically modified or deleted, wherein the sequence maintains the CD40-L binding properties of the sequence specified in (i).

5. A pharmaceutical composition according to claim 1, wherein up to 5% of the amino acids have been replaced, chemically modified or deleted, wherein the sequence maintains the CD40-L binding properties of (i).

6. A pharmaceutical composition according to claim 1, wherein in the chimeric protein of (iv), the amino acid sequence of (i), (ii) or (iii), is conjugated to an entity selected from: an antibody or an antibody fragment, a glycoprotein, a fragment of the comp protein b-zip

7. A pharmaceutical composition according to claim 6, wherein said antibody fragment is the Fc region of an antibody of IgG

8. A pharmaceutical composition according of claim 1, wherein in (i), (ii), (iii) or (iv), up to 20% of the amino acid of the native sequence has been replaced with a naturally or non-naturally occurring amino acid or with a peptidomimetic organic moiety; and/or up to 20% of the amino acids have their side chains chemically modified and/or up to 20% of the amino acids have been deleted, provided that at least 80% of the amino acids in the parent sequence of (i), (ii), (iii) or (iv) are maintained unaltered, and provided that the amino acid maintains the biological activity of the parent sequence of (i), (ii), (iii) or (iv).

9. A pharmaceutical composition according of claim 1, wherein in (i), (ii), (iii), or (iv), at least one of the amino acids is replaced by the corresponding D-amino acid.

10. A pharmaceutical composition according of claim 1, wherein in (i), (ii), (iii), or (iv) the peptidic backbone of at least one of the amino acids has been altered to a non-naturally occurring peptidic backbone.

11. A pharmaceutical composition according to claim 1 (ii), where the fragment comprises amino acids 166-203 of SEQ ID No. 1.

12. A pharmaceutical composition according to claim 1 further comprising an antibody capable of selectively binding to the amino acid of SEQ ID NO:1 while essentially not binding to wild-type soluble CD40.

13. A nucleic acid molecule encoding the agent of claim 1.

14. A molecule that hybridizes with the nucleic acid molecule of claim 13 under stringent hybridization conditions.

15. A molecule according to claim 14, wherein the molecule is selected from the group consisting of a nucleic acid and a peptide nucleic acid.

16. A molecule according to claim 15, wherein the molecule is selected from the group consisting of an antisense nucleic acid, a ribozyme, a DNAzyme, an RNAi, and a triple helix-forming nucleic acid.

17. A vector comprising the nucleic acid of claim 13.

18. A host cell comprising the vector of claim 17.

19. The host cell of claim 17, wherein the host cell is a mammalian cell.

20. The host cell of claim 17, wherein the host cell is a bacterial cell.

21. A pharmaceutical composition comprising a pharmaceutically acceptable carrier and an antibody capable of selectively binding to the amino acid of SEQ ID NO:1, while essentially not binding to wild-type soluble CD40.

22. A method of identifying a CD40 splice variant polypeptide binding partner in a sample, the method comprising:
incubating a heparanase splice variant polypeptide with a sample; and
identifying a component in the sample that binds to the heparanase splice variant polypeptide.

23. A method of identifying an agent that interferes with the binding of a CD40 splice variant polypeptide with its binding partner, the method comprising:
incubating a CD40 splice variant polypeptide and a CD40 splice variant polypeptide binding partner with an agent; and
detecting decreased binding between the CD40 splice variant polypeptide and the binding partner, wherein a decrease in the binding compared to a control reaction is indicative that the agent interferes with the binding.

24. A method of identifying an agent that alters the gene expression of a CD40 splice variant polypeptide in a cell, the method comprising the steps of:
treating a cell that expresses a CD40 splice variant polypeptide with an agent; and
measuring the gene expression of the CD40 splice variant polypeptide.

25. The method of claim 24, wherein the measuring step comprises measuring the transcription of a CD40 splice variant nucleic acid or fragment thereof.

26. The method of claim 24, wherein the agent is an agonist.

27. The method of claim 25, wherein the agent is an antagonist.

28. A method of identifying the presence of a CD40 splice variant polypeptide in a sample, the method comprising the step of detecting binding of the CD40 splice variant polypeptide with a specific antibody.

29. A method of identifying the presence of a CD40 splice variant polypeptide in a sample, the method comprising the steps of:
(a) amplifying a CD40 splice variant RNA to create an amplification product; and
(b) detecting levels of the amplification product.

30. A method of producing a polyclonal antibody or antibody fragment that binds to a CD40 splice variant polypeptide, the method comprising the steps of:
inoculating a non-human animal with a CD40 splice variant polypeptide; and
isolating the antibody from the animal.

31. A method of producing a monoclonal antibody or antibody fragment that binds to a CD40 splice variant polypeptide, the method comprising the steps of:
culturing in culture fluid a cell that produces a monoclonal antibody or antibody fragment that binds to a CD40 splice variant polypeptide or fragment thereof; and
isolating the antibody from the cell or the culture fluid.

32. A method of treating a condition associated with aberrant levels of CD40, the method comprising the step of increasing the levels of a CD40 splice variant.

33. The method of claim 32, wherein the increasing step comprises increasing the gene expression of the CD40 splice variant.

34. The method of claim 32, wherein the increasing step comprises administering a clinically effective amount of the CD40 splice variant.

35. The method of claim 32, wherein the CD40 splice variant is an agent comprising the amino acid sequence selected from the group consisting of:
(i) the amino acid sequence depicted in SEQ. ID No. 1;
(ii) a fragment of at least 10 amino acids of the amino acid sequence of (i), having at least four consecutive amino acids of the segment 166-203 of SEQ. ID No. 1, said fragment still having CD40-L binding properties substantially as those of the sequence of (i);
(iii) a variant of the amino acid sequence of (i) or (ii) wherein up to 20% of the amino acids have been replaced, chemically modified or deleted, wherein the sequence substantially maintains the CD40-L binding properties of (i); and
(iv) a chimeric protein comprising the amino acid sequence of (i), (ii) or (iii), conjugated to another entity.

36. The method of claim 35 wherein said agent is (i).

37. A method for treating a disease, wherein a beneficial therapeutic effect is achieved by the interruption of the CD40-R-CD40-L interaction, comprising administering to an individual in need of such treatment a therapeutically effective amount of a composition selected from a composition as defined by claim 1.

38. The method according to claim 37, wherein the disease is selected from graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, asthma, cryoglobulinemia, primary biliary sclerosis and pernicious anemia, end-stage fibrosis, hyperoxic injuries, radiation-induced injuries, cancers, epithelial neoplasia, nasopharyngeal carcinoma, osteosarcoma, neuroblastoma and bladder carcinoma, AIDS-related lymphoma, impaired renal function, including chronic renal failure, haemodialysis and chronic ambulatory peritoneal dialysis (CAPD) patients.

39. A method for treating a disease wherein a beneficial therapeutic effect is achieved by the interruption of the CD40-R-CD40-L interaction, comprising administering to an individual in need of such treatment a therapeutically effective amount of a composition of claim 12.

40. The method according to claim 39, wherein the disease is selected from graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, asthma, cryoglobulinemia, primary biliary sclerosis and pernicious anemia, end-stage fibrosis, hyperoxic injuries, radiation-induced injuries, cancers, epithelial neoplasia, nasopharyngeal carcinoma, osteosarcoma, neuroblastoma and bladder carcinoma, AIDS-related lymphoma, impaired renal function, including chronic renal failure, haemodialysis and chronic ambulatory peritoneal dialysis (CAPD) patients.

41. A method for treating a disease wherein a beneficial therapeutic effect is achieved by the interruption of the CD40-R-CD40-L interaction, comprising administering to an individual in need of such treatment a therapeutically effective amount of a composition of claim 13.

42. The method according to claim 41, wherein the disease is selected from graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, inflammatory bowel diseases (IBD), ulcerative colitis, Crohn's disease, hematological malignancies, Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Lupus (SLE), Grave's disease, myasthenia gravis, autoimmune hemolytic anemia, autoimmune thrombocytopenia, asthma, cryoglobulinemia, primary biliary sclerosis and pernicious anemia, end-stage fibrosis, hyperoxic injuries, radiation-induced injuries, cancers, epithelial neoplasia, nasopharyngeal carcinoma, osteosarcoma, neuroblastoma and bladder carcinoma, AIDS-related lymphoma, impaired renal function, including chronic renal failure, haemodialysis and chronic ambulatory peritoneal dialysis (CAPD) patients.

43. A method for treating a subject receiving a transplant, comprising administering the pharmaceutical composition of claim 1 to the subject.

44. A method according to claim 43, wherein said CD40 splice variant comprises skipping 6 variant.

45. A method according to claim 43, further comprising administering at least one additional therapeutic agent to the subject.

46. A method according to claim 43, wherein said additional therapeutic agent comprises a small molecule.

47. A method according to claim 46, wherein said additional therapeutic agent comprises an antibody.

48. A method according to claim 47, wherein said additional therapeutic agent comprises a protein bound to an Fc fragment.

49. A method according to claim 45, wherein said additional therapeutic agent comprises DST.

50. A method according to claim 43, wherein said CD40 splice variant is fused to Fc.

51. A method for detecting the presence exon 6 skipping expression in a sample the method comprising detecting the presence of mRNA coding for the amino acid sequence of SEQ ID No: 1 in the sample, wherein the presence of said mRNA indicates exon skipping 6 is expressed in said sample.

52. The method according to claim 51 wherein the detection of the mRNA is by amplification.

53. A method according to claim 52, wherein the amplification is by PCR.

54. A method according to claim 51, wherein the detection is by hybridization with a detectable nucleic acid probe for the mRNA.

55. A method according to claim 51, wherein the sample is selected from: a tissue sample, body fluid, cell culture, in situ tissue, in vivo tissue.

56. A method according to claim 51, wherein expression is quantified by comparing the expression results with a predetermined calibration curve.

57. A method according to claim 51, wherein said method is used to detect a condition characterized by overexpression of SEQ ID No. 1 (exon 6 skipping).

58. A method according to claim 51, wherein said method is used to detect a condition characterized by underexpression of SEQ ID No. 1 (exon 6 skipping).

59. A method for detecting the presence exon 6 skipping expression in a sample the method comprising determining the presence the amino acid sequence of SEC ID No: 1 in the sample, wherein a positive determination indicating the expression of exon skipping 6.

60. A method according to claim 59, wherein the determination is by an antibody which selectively binds to the amino acid sequence of SEQ ID No: 1.

61. A method according to claim 59, wherein the antibody does not bind to WT CD40.

62. A method according to claim 59, wherein the sample is selected from: a tissue sample, body fluid, cell culture, in situ tissue, in vivo tissue.

63. A method according to claim 59, wherein expression is quantified by comparing the expression results with a predetermined calibration curve.

64. A method according to claim 59, wherein said method is used to detect a condition characterized by overexpression of SEQ ID No. 1 (exon 6 skipping).

65. A method according to claim 59, wherein said method is used to detect a condition characterized by underexpression of SEQ ID No. 1 (exon 6 skipping).


SEQUENCE LISTING (31 sequences)

SEQ ID NO: 1 - protein, 203 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTRSPGSAESPGGDPHHLRDPVCHPLGAGLYQKGGQEANQ

SEQ ID NO: 2 - DNA, 759 bp, Homo sapiens
tggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggagg

SEQ ID NO: 3 - protein, 15 aa, Artificial Sequence
  Description of Artificial Sequence synthetic peptide
ESPGGDPHHLRDPVC

SEQ ID NO: 4 - DNA, 5234 bp, Artificial Sequence
  Description of Artificial Sequence plasmid vector pTen21
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatccaagcttaagagctcgagcggccgcctgcagccggtacccagatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 5 - DNA, 32 bp, Artificial Sequence
  Description of Artificial SequencePCR primer
actagatatcatggttcgtctgcctctgcagt

SEQ ID NO: 6 - DNA, 30 bp, Artificial Sequence
  Description of Artificial SequencePCR primer
aagcagatcttatctcagccgatcctgggg

SEQ ID NO: 7 - DNA, 21 bp, Artificial Sequence
  Description of Artificial SequencePCR primer
gcatttgaggatgccgggacc

SEQ ID NO: 8 - DNA, 20 bp, Artificial Sequence
  Description of Artificial SequencePCR primer
cataatcaaagaatcgtacg

SEQ ID NO: 9 - DNA, 651 bp, Artificial Sequence
  Description of Artificial Sequence Clone 7 of pTen21-CD40wtEC
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagataagatctgggtaaacgcagttccaagtaaatgaatcgtttttaaaataacaaatcaattgttttataatat

SEQ ID NO: 10 - DNA, 20 bp, Artificial Sequence
  Description of Artificial SequencePCR primer
caccatctgcacctgtgaag

SEQ ID NO: 11 - DNA, 30 bp, Artificial Sequence
  Description of Artificial SequencePCR primer
aatgagatcttattggttggcttcttggcc

SEQ ID NO: 12 - DNA, 5775 bp, Artificial Sequence
  Description of Artificial Sequence plasmid vector pTen21-CD40wtEC
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagataagatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 13 - DNA, 5805 bp, Artificial Sequence
  Description of Artificial Sequence plasmid vector pTen21-CD40_Skip6
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaataagatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 14 - DNA, 810 bp, Artificial Sequence
  Description of Artificial SequenceClone 5 of pTen21-CD40_Skip6
ccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaataagatctgggtaaacgcagttccaagtaaatgaatcgtttttaaaataacaaatcaattgttttataata

SEQ ID NO: 15 - DNA, 5909 bp, Artificial Sequence
  Description of Artificial Sequenceplasmid vector pTen21-Fc
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatccaagcttaagagctcgagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 16 - DNA, 914 bp, Artificial Sequence
  Description of Artificial SequenceClone 19 of pTen21-Fc
aacccaacacaatatattatagttaaataagaattattatcaaatcatttgtatattaattaaaatactatactgtaaattacatttttatttacaatcagctgaaagcctaacgcagatcggatccgatatccaagcttaagagctcgagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttata

SEQ ID NO: 17 - DNA, 40 bp, Artificial Sequence
  Description of Artificial Sequence PCR primer
cacaagatctgggctctacgtatctcagccgatcctgggg

SEQ ID NO: 18 - DNA, 6484 bp, Artificial Sequence
  Description of Artificial Sequenceplasmid vector pTen21-CD40wtEC-Fc
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 19 - DNA, 1477 bp, Artificial Sequence
  Description of Artificial SequenceClone 37 of pTen21-CD40wtEC-Fc
ccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaat

SEQ ID NO: 20 - DNA, 41 bp, Artificial Sequence
  Description of Artificial SequencePCR primer
cacaagatctgggctctacgtattggttggcttcttggcca

SEQ ID NO: 21 - DNA, 6514 bp, Artificial Sequence
  Description of Artificial Sequenceplasmid vector pTen21-CD40_Skip6-Fc
aagctggacgtccagcttacaatatgactcatttgtctttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcacaactaaactcgctttacgggtagaattctacgcgtaaaacatgattgataattaaataattcatttgcaagctatacgttaaatcaaacggacgtcatggaattgtataatattaaatatgcaattgatccaacaaataaaattgtaatagagcaagtcgactatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaagcgaagcaaaaactgcgcattggagcaccaatttttgccagcaaaaagctggcttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctagagcaagaaaataaaacgcccaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataaggtaccggatctgggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccataattatttgattaaaattgttgttttctttgttcatatacaatagtgtgttttgcctaaacgtgtactgcataaactccatgcgagtgtatagcgagctagtggctaacgcttgccccactaaagtagattcgtcaaaatcctcaatttcatcaccctcctccaagtttaacatttggccgtcggaattaacttctaaagatgccacataatctaataaatgaaatagagattcaaacgtggcgtcgtcgtccgtttcgaccatttccgaaaagaactcgggcataaactctatgatttctctagacgtggtgttgtcgaaactctcaaagtatgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgcgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcaaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 22 - DNA, 915 bp, Artificial Sequence
  Description of Artificial SequenceClone 33 of pTen21-CD40_Skip6-Fc
tttaattcaacccaacacaatatattatagttaaataagaattattatcaaatcatttgtatattaattaaaatactatactgtaaattacatttttatttacaatcagctgaaagcctaacgcagatcggatccgatatcatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaatacgtagagcccagatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccac

SEQ ID NO: 23 - protein, 165 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWT

SEQ ID NO: 24 - protein, 38 aa, Homo sapiens
RSPGSAESPGGDPHHLRDPVCHPLGAGLYQKGGQEANQ

SEQ ID NO: 25 - DNA, 58 bp, Artificial Sequence
  Description of Artificial Sequencemultiple cloning site of pTen21 plasmid
ggatccgatatccaagcttaagagctcgagcggccgcctgcagccggtacccagatct

SEQ ID NO: 26 - DNA, 15 bp, Artificial Sequence
  Description of Artificial SequenceDescription of Artificial Sequence pTen21 vector fragment following enzyme digestion.
ggatccgatatcatg

SEQ ID NO: 27 - DNA, 15 bp, Artificial Sequence
  Description of Artificial Sequence pTen21-CD40wtEC clone 7 vector fragment following enzyme digestion.
ggatccgatatcatg

SEQ ID NO: 28 - DNA, 15 bp, Artificial Sequence
  Description of Artificial Sequence pTen21-Fc clone 19 vector fragment following enzyme digestion.
ggatccgatatcatg

SEQ ID NO: 29 - DNA, 15 bp, Artificial Sequence
  Description of Artificial SequencePCR fragment of CD40wtEC digested with restriction enzymes.
taaggtaccggatct

SEQ ID NO: 30 - DNA, 15 bp, Artificial Sequence
  Description of Artificial Sequence pTen21-CD40wtEC-Fc clone 37 vector fragment following enzyme digestion.
ggatccgatatcatg

SEQ ID NO: 31 - DNA, 15 bp, Artificial Sequence
  Description of Artificial SequencePCR fragment of CD40-skipping 6 following restriction enzyme digestion.
taaggtaccggatct